(19) United States
(12) Patent Application Publication
(10) Pub. No.: US 2025/0091984 A1
(43) Pub. Date: Mar. 20, 2025

(54) FRAGRANCE AND FLAVOR MATERIALS

(71) Applicant: TAKASAGO INTERNATIONAL CORPORATION, Tokyo (JP)

(72) Inventors: Thalia S. Dickerson, Haskell, NJ (US); Robert Tureski, Edgewater, NJ (US); Maureen Blandino, Dumont, NJ (US)

(73) Assignee: TAKASAGO INTERNATIONAL CORPORATION, Tokyo (JP)

(21) Appl. No.: 18/843,220

(22) PCT Filed: Apr. 7, 2023
(86) PCT No.: PCT/US2023/017919

§ 371 Date: Aug. 30, 2024

Related U.S. Application Data
(60) Provisional application No. 63/328,669, filed on Apr. 7, 2022.

(51) Int. Cl. C07C 251/54; A23L 27/20; C07C 251/50; C07C 251/56; C11B 9/00

(52) U.S. Cl. CPC C07C 251/54; A23L 27/203; C07C 251/50; C07C 251/56; C11B 9/0034

(57) ABSTRACT

The present disclosure is directed to the synthesis and application of novel compounds having unique and desired flavor and/or fragrance characteristics. The compounds of the present disclosure can be employed alone or incorporated as fragrance or flavor compounds in fragrance or flavor compositions. The present disclosure is also directed to consumer products comprising such fragrance or flavor compounds and/or fragrance or flavor compositions.

### CROSS-REFERENCE TO RELATED APPLICATIONS

This application claims the benefit of priority to U.S. Provisional Patent Application Ser. No. 63/328,669 filed on Apr. 7, 2022, the contents of which are incorporated herein by reference in their entirety.

### FIELD

The present application relates to compounds useful as fragrance or flavor components in fragrance or flavor compositions.

### BACKGROUND

There is a continuing interest in the preparation of synthetic fragrance and flavor compounds and their use in consumer products. Unique fragrance and flavor compounds are needed for fragrance and flavor compositions with pleasing and consumer preferred odor and taste profiles for use in a variety of consumer products. One strategy to preparing such novel compounds is to apply a synthetic chemical reaction to readily available substrates.

### SUMMARY

The present disclosure is directed to the synthesis and application of novel oximes and derivatives thereof having unique and desired organoleptic characteristics. The compounds of the present disclosure can be employed alone or in combination with additional compounds. The compounds of the present disclosure can be useful as fragrance or flavor components incorporated into fragrance or flavor compositions.

In certain embodiments, the presently disclosed subject matter provides a compound of Formula (I), or isomer thereof:

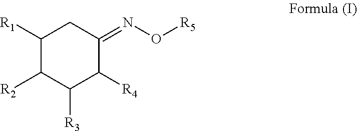

wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group;
wherein if R₄ is a sec-butyl group, R₅ is not a hydrogen atom; and
wherein if R₁ and R₃ are a methyl group, R₅ is not an ethyl group.

In certain embodiments, the compound of Formula (I) is selected from the group consisting of 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, 2-(sec-butyl)cyclohexan-1-one O-isobutyryl oxime, 3,5-dimethylcyclohexan-1-one oxime, 3,5-dimethylcyclohexan-1-one O-allyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, 3,5-dimethylcyclohexan-1-one O-cyclohexanecarbonyl oxime, 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the presently disclosed subject matter provides a compound of Formula (II), or isomer thereof:

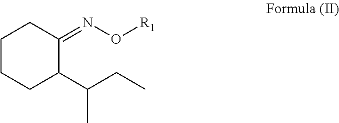

wherein
R₁ is a C1-C5 linear or branched alkyl group, C2-C5 linear or branched alkenyl group, C1-C5 linear or branched alcohol group, or C(O)R₂, and
R₂ is a C1-C5 linear or branched alkyl group.

In certain embodiments, the compound of Formula (II) is selected from the group consisting of 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, and 2-(sec-butyl)cyclohexan-1-one O-isobutyryl oxime.

In certain embodiments, the presently disclosed subject matter provides a compound of Formula (III), or isomer thereof:

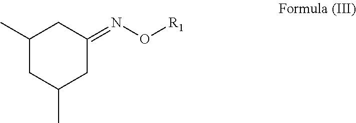

wherein
R₁ is a hydrogen atom, methyl group, C3-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (III) is selected from the group consisting of 3,5-dimethylcyclohexan-1-one oxime, 3,5-dimethylcyclohexan-1-one O-allyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, and 3,5-dimethylcyclohexan-1-one O-cyclohexanecarbonyl oxime.

In certain embodiments, the presently disclosed subject matter provides a compound of Formula (IV), or isomer thereof:

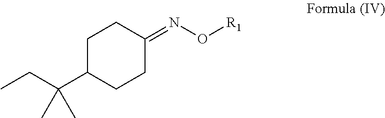

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (IV) is selected from the group consisting of 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the presently disclosed subject matter provides a flavor or fragrance composition comprising a compound of Formula (I), or isomer thereof:

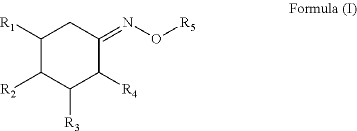

wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group.

In certain embodiments, the compound of Formula (I) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, 2-(sec-butyl)cyclohexan-1-one O-isobutyryl oxime, 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, 3,5-dimethylcyclohexan-1-one O-cyclohexanecarbonyl oxime, 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the presently disclosed subject matter provides a flavor or fragrance composition comprising a compound of Formula (II), or isomer thereof:

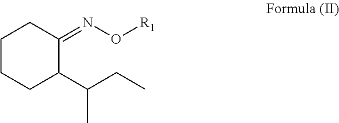

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.

In certain embodiments, the compound of Formula (II) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one 0-propionyl oxime, and 2-(sec-butyl)cyclohexan-1-one O-isobutyryl oxime.

In certain embodiments, the presently disclosed subject matter provides a flavor or fragrance composition comprising a compound of Formula (III), or isomer thereof:

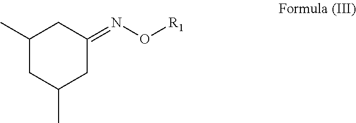

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (III) is selected from the group consisting of 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, and 3,5-dimethylcyclohexan-1-one O-cyclohexanecarbonyl oxime.

In certain embodiments, the presently disclosed subject matter provides a flavor or fragrance composition comprising a compound of Formula (IV), or isomer thereof:

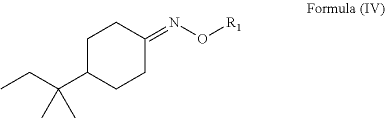

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (IV) is selected from the group consisting of 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the flavor or fragrance composition comprises at least one compound of Formula (I), Formula (II), Formula (III), or Formula (IV).

In certain embodiments, the flavor or fragrance composition is a fragrance composition. In particular embodiments, the fragrance composition comprises at least one compound in a concentration of from about 0.001% to about 20% by weight of the fragrance composition. In certain embodiments, the fragrance composition comprises at least one compound in a concentration of from about 0.01% to about 10% by weight of the fragrance composition. In certain embodiments, the fragrance composition further comprises at least one compounds selected from the group consisting of one or more aldehydic compound(s), animalic compound(s), balsamic compound(s), citrus compound(s), floral compound(s), fruity compound(s), gourmand compound, green compound(s), herbaceous compound(s), marine compound(s), mossy compound(s), musk compound(s), piney compound(s), powdery compound(s), spicy compound(s), and/or woody/amber compound(s).

In certain embodiments, the fragrance composition further comprises a fragrance carrier or a fragrance base. In particular embodiments, the fragrance composition can be in the form of a simple mixture or a biphasic system. In certain embodiments, the biphasic system is an emulsion or microemulsion.

In certain embodiments, the fragrance composition can be used in consumer products selected form the group consisting of air care products, fine fragrances, personal care products, cosmetic, fabric and home care products, pharmaceutical and over-the-counter products, insect repellents or insecticides, and pet care products

In certain embodiments, the flavor or fragrance composition is a flavor composition. In particular embodiments, the flavor composition comprises at least one compound in a concentration of from about 0.0005% to about 20% by weight of the flavor composition. In certain embodiments, the flavor composition comprises at least one compound in a concentration of from about 0.0005% to about 10% by weight of the flavor composition.

In certain embodiments, the flavor composition further comprises at least one additional flavor composition or flavor compound. In particular embodiments, the flavor composition further comprises at least one solvent. In certain embodiments, flavor composition further comprises a flavor carrier. In particular embodiments, the flavor composition further comprises at least one material selected from the group consisting of one or more of solvents, support materials, diluents, carriers, preservatives, flavor compounds, flavor enhancers, antioxidants, color retention agents, dyes or lakes, sequestrants, emulsifiers, stabilizers, acids, bases, and anticaking agents.

In certain embodiments, the flavor composition can be in a simple mixture or an encapsulated form. In particular embodiments, the simple mixture is a mixture of flavor compounds and solvents.

In certain embodiments, the flavor composition can be used in consumer products selected from the group consisting of foods or beverages, oral care products, animal feed and pet foods, pharmaceuticals, and sexual health care products.

In certain embodiments, the presently disclosed subject matter provides a method of modifying the odor properties of a fragrance composition by adding to the fragrance composition an olfactory effective quantity of a fragrance compound of Formula (I), or isomer thereof:

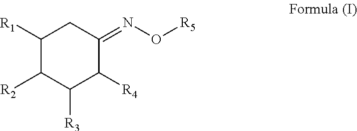

wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group.

In certain embodiments, the compound of Formula (I) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, 2-(sec-butyl)cyclohexan-1-one O-isobutyryl oxime, 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, 3,5-dimethylcyclohexan-1-one O-cyclohexanecarbonyl oxime, 4-(tert-pentyl)cyclohexan-1-one oxime, 4-(tert-pentyl)cyclohexan-1-one O-methyl oxime, 4-(tert-pentyl)cyclohexan-1-one 0-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the presently disclosed subject matter provides a method of modifying the odor properties of a fragrance composition by adding to the fragrance composition an olfactory effective quantity of a fragrance compound of Formula (II), or isomer thereof:

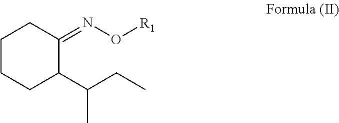

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.

In certain embodiments, the compound of Formula (II) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one 0-propionyl oxime, and 2-(sec-butyl)cyclohexan-1-one O-isobutyryl oxime.

In certain embodiments, the presently disclosed subject matter provides a method of modifying the odor properties of a fragrance composition by adding to the fragrance composition an olfactory effective quantity of a fragrance compound of Formula (III), or isomer thereof:

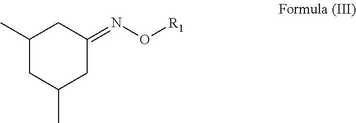

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (III) is selected from the group consisting of 3,5-dimethylcyclohexan-1-one oxime, 3,5-dimethylcyclohexan-1-one 0-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, and 3,5-dimethylcyclohexan-1-one O-cyclohexanecarbonyl oxime.

In certain embodiments, the presently disclosed subject matter provides a method of modifying the odor properties of a fragrance composition by adding to the fragrance composition an olfactory effective quantity of a fragrance compound of Formula (IV), or isomer thereof:

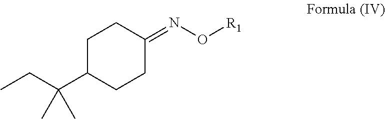

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In certain embodiments, the compound of Formula (IV) is selected from the group consisting of 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

The foregoing has outlined broadly the features and technical advantages of the present application in order that the detailed description that follows can be better understood. Additional features and advantages of the application will be described hereinafter which form the subject of the claims of the application. It should be appreciated by those skilled in the art that the conception and specific embodiments disclosed can be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### DETAILED DESCRIPTION

As noted above, there remains a need and demand in the art for unique fragrance and flavor components and fragrance or flavor compositions with pleasing and consumer preferred fragrance or flavor attributes. There is also a need for more efficient synthesis techniques to prepare such fragrance and flavor components. The presently disclosed subject matter addresses these needs through the compounds disclosed herein and/or new fragrance and flavor compositions comprising one or more of the disclosed compounds.

For clarity, and not by way of limitations, the detailed description is divided into the following subsections:
1. Definitions;
2. Oximes and Derivatives Thereof;
3. Synthesis of Oximes and Derivatives Thereof, and
4. Compositions.

### 1. Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compounds, compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “weight percent” or “wt-%” is meant to refer to the quantity by weight of a component in a material as a percentage of the total wet weight of the material (i.e., a fragrance formulation). Unless otherwise specified, all amounts expressed as a percentage herein represent the amount in weight percent.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Also, as used herein, the terms “constitutional isomers” refers to different compounds which have the same numbers of, and types of, atoms but the atoms are connected differently. Therefore, as used herein, isomers can include constitutional isomers, enantiomers, diastereomers, or racemic mixtures of the compound.

As used herein, the term “fragrance composition” refers to a mixture comprising one or more of presently disclosed fragrance compounds, and optionally other fragrance compound(s) in any of their forms, and optionally one or more solvents. As known in the art, a fragrance composition contains one or more fragrance compounds in order to impart an olfactory note to the end product formulation (e.g., a household cleaner, perfume, or other consumer product) to which it is added. In one embodiment, the fragrance composition contains two or more fragrance compounds which, collectively and in combination with the solvent to which they are added, impart an intended olfactory note (e.g., a hedonically pleasing “tropical” note) to a human in close proximity to the fragrance composition. In general terms, fragrance compounds belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin, and are known to perfumists of ordinary skill in the art. Many of these fragrance compounds are listed in reference texts such as S. Arctander, Perfume and Flavor Chemicals, 1969, Montclair, New Jersey, USA or any of its more recent versions, each of which are hereby incorporated by reference. The skilled person in the art of perfumery is able to select fragrance compounds from different categories, and solvents to create fragrance compositions on the basis of his or her general knowledge and according to the nature of the intended end product and desired scent.

As used herein, the term “accord” refers to a composition that contains one or more different compounds that creates a specific smell, odor or scent. One or more “accords” can be utilized as part of fragrance composition.

As used herein, the term “flavor composition” refers to a composition that contains one or more of presently disclosed flavor compound(s), and optionally other flavor compound(s) that provide a desired taste when combined with a solvent that is suitable for oral administration and oral consumption. Examples of other flavor compounds that are generally included in a flavor composition are listed in S. Arctander, Perfume and Flavor Chemicals, 1969, Montclair, New Jersey, USA. The skilled person in the art of flavors is able to select them on the basis of its general knowledge and according to the nature of the consumer product to be flavored and the desired taste.

As used herein, the phrase “consumer product” or “end product” refers to composition that is in a form ready for use by the consumer for the marketed indication. A solvent suitable for use in a consumer product is a solvent that, when combined with other components of the consumer product, will not render the consumer product unfit for its intended consumer use.

As used herein, the term “improving” or “improve” is understood to mean raising a fragrance composition to a more desirable character, the term “enhancing” or “enhance” is understood to mean making the fragrance composition greater in effectiveness, such as strength, and the term “modifying” is understood to mean providing the fragrance composition with a change in character.

### 2. Oximes and Derivatives Thereof

The present disclosure is directed to the synthesis and application of novel oximes and derivatives thereof having unique and desired fragrance and/or flavor characteristics.

In certain embodiments, the presently disclosed subject matter provides compounds of the Formula (I),

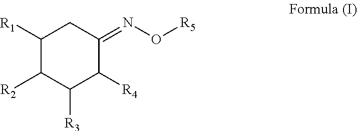

in which
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group.

In alternative embodiments, the presently disclosed subject matter provides compounds of the Formula (I) wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group;
wherein if R₄ is a sec-butyl group, R₅ is not a hydrogen atom.

In alternative embodiments, the presently disclosed subject matter provides compounds of the Formula (I) wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C2-C5 linear or branched alcohol group, or C(O)R₆, and
R₆ is a C1-C5 linear or branched alkyl group or C3-C6 substituted or unsubstituted cycloalkyl group;
wherein if R₁ and R₃ are a methyl group, R₅ is not an ethyl group.

In alternative embodiments, the presently disclosed subject matter provides compounds of the Formula (I) wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group;
wherein if R₄ is a sec-butyl group, R₅ is not a hydrogen atom; and
wherein if R₁ and R₃ are a methyl group, R₅ is not an ethyl group.

Non-limiting examples of suitable C5 branched alkyl groups at R₂ include, but are not limited to, a pentyl group, a 2-methylbutan-2-yl group, a 2,2-dimethylpropyl group, a 3-methylbutyl group, a pentan-2-yl group, a pentan-3-yl group, a 3-methylbutan-2-yl group, and a 2-methylbutyl group.

Non-limiting examples of suitable C1-C5 linear or branched alkyl groups at R₅ and/or R₆ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₅ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl groups at R₆ include, but are not limited to, a cyclopropyl group, a cyclopentyl group, and a cyclohexyl group.

Non-limiting examples of suitable C1-C5 linear or branched alcohol groups at R₅ include, but are not limited to, a methyl alcohol, an ethyl alcohol, an n-propyl alcohol, an isopropyl alcohol, and an n-butyl alcohol.

In certain embodiments, R₁ is a hydrogen atom or methyl group.

In certain embodiments, R₂ is a hydrogen atom or a 2-methylbutan-2-yl group.

In certain embodiments, R₃ is a hydrogen atom or methyl group.

In certain embodiments, R₄ is a hydrogen atom or sec-butyl group.

In certain embodiments, R₅ is a hydrogen atom, a methyl group, an ethyl group, an n-butyl group, an allyl group, a 3-methyl-2-butenyl group, an n-propyl alcohol group, or a C(O)R₆ group.

In certain embodiments, R₆ is a methyl group, an ethyl group, an isopropyl group, a cyclopropyl group, or a cyclohexyl group.

In certain embodiments, if R₄ is a sec-butyl group, R₅ is not a hydrogen atom.

In certain embodiments, if R₁ and R₃ are a methyl group, R₅ is not an ethyl group.

In certain embodiments, the compound of Formula (I) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, 2-(sec-butyl)cyclohexan-1-one O-isobutyryl oxime, 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, 3,5-dimethylcyclohexan-1-one O-cyclohexanecarbonyl oxime, 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the compounds include constitutional isomers, enantiomers, stereoisomers, and racemic mixtures of said compounds listed herein.

In certain embodiments, the presently disclosed subject matter provides compounds of the Formula (II),

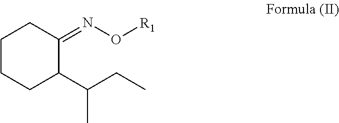

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.

In alternative embodiments, the presently disclosed subject matter provides compounds of the Formula (II), wherein
R₁ is a C1-C5 linear or branched alkyl group, C2-C5 linear or branched alkenyl group, C1-C5 linear or branched alcohol group, or C(O)R₂, and
R₂ is a C1-C5 linear or branched alkyl group.

Non-limiting examples of suitable C1-C5 linear or branched alkyl groups at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C1-C5 linear or branched alcohol groups at R₁ include, but are not limited to, a methyl alcohol, an ethyl alcohol, an n-propyl alcohol, an isopropyl alcohol, and an n-butyl alcohol.

In certain embodiments, R₁ is a methyl group, an ethyl group, an n-butyl group, an allyl group, a 3-methyl-2-butenyl group, an n-propyl alcohol group, or a C(O)R₂ group.

In certain embodiments, R₂ is a methyl group, an ethyl group, or an isopropyl group.

In certain embodiments, the compound of Formula (II) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, and 2-(sec-butyl)cyclohexan-1-one O-isobutyryl oxime.

In certain embodiments, the compounds include constitutional isomers, enantiomers, stereoisomers, and racemic mixtures of said compounds listed herein.

In certain embodiments, the presently disclosed subject matter provides compounds of the Formula (III),

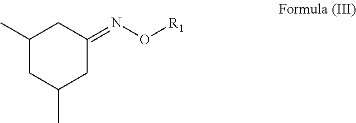

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

In alternative embodiments, the presently disclosed subject matter provides compounds of the Formula (III) wherein
R₁ is a hydrogen atom, methyl group, C3-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

Non-limiting examples of suitable C1-C5 linear or branched alkyl group at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl group at R₂ include, but are not limited to, a cyclopropyl group, a cyclopentyl group, and a cyclohexyl group.

Non-limiting examples of suitable C1-C5 linear or branched alcohol group at R₁ include, but are not limited to, a methyl alcohol, an ethyl alcohol, an n-propyl alcohol, an isopropyl alcohol, and an n-butyl alcohol.

In certain embodiments, R₁ is a hydrogen atom, an allyl group, an n-propyl alcohol group, or a C(O)R₂ group.

In certain embodiments, R₂ is a methyl group, an ethyl group, an isopropyl group, a cyclopropyl group, or a cyclohexyl group.

In certain embodiments, the compound of Formula (III) is selected from the group consisting of 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, and 3,5-dimethylcyclohexan-1-one O-cyclohexanecarbonyl oxime.

In certain embodiments, the compounds include constitutional isomers, enantiomers, stereoisomers, and racemic mixtures of said compounds listed herein.

In certain embodiments, the presently disclosed subject matter provides compounds of the Formula (IV),

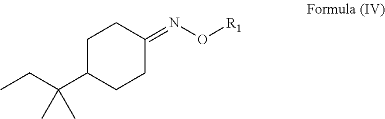

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

Non-limiting examples of suitable C1-C5 linear or branched alkyl group at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl group at R₂ include, but are not limited to, a cyclopropyl group, a cyclopentyl group, and a cyclohexyl group.

Non-limiting examples of suitable C1-C5 linear or branched alcohol group at R₁ include, but are not limited to, a methyl alcohol, an ethyl alcohol, an n-propyl alcohol, an isopropyl alcohol, and an n-butyl alcohol.

In certain embodiments, R₁ is a hydrogen atom, a methyl group, an allyl group, or a C(O)R₂ group.

In certain embodiments, R₂ is a methyl group, an ethyl group, an isopropyl group, a cyclopropyl group, or a cyclohexyl group.

In certain embodiments, the compound of Formula (IV) is selected from the group consisting of 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl, and 4-(tert-pentyl)cyclohexan-1-one O-cyclohexanecarbonyl oxime.

In certain embodiments, the compounds include constitutional isomers, enantiomers, stereoisomers, and racemic mixtures of said compounds listed herein.

### 3. Synthesis of Oximes and Derivatives Thereof

The presently disclosed compounds can be prepared synthetically through various chemical transformations.

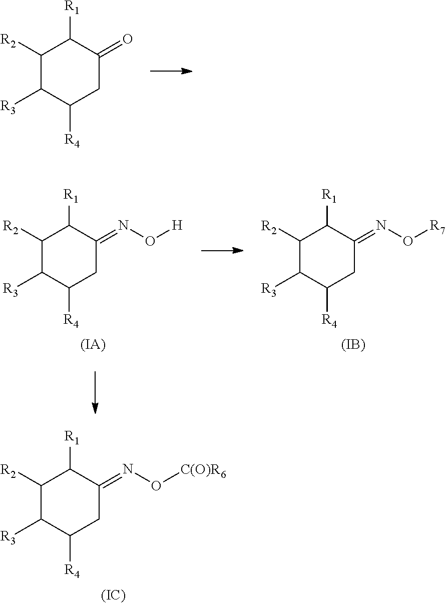

For the synthesis of Formula (IA), oximation of a ketone with hydroxylamine, in a salt form, for example, but not limited to, hydrochloride salt, in the presence of a base, for example, but not limited to, potassium hydroxide, sodium carbonate, pyridine, or triethylamine, is a preferred method. Other conditions include, but are not limited to, reacting a ketone with hydroxylamine in the presence of an acid, for example, but not limited to, sulfuric acid or triflic acid, or with a masked hydroxylamine, for example, but not limited to, N,O-bis(trimethylsilyl)hydroxylamine, in the presence of a strong base, for example, but not limited to, potassium hydride, sodium hydride, or lithium diisopropylamide, or an ammoximation reaction where an amine, for example, but not limited to, ammonia, reacts with a peroxide, for example, but not limited to, hydrogen peroxide, in the presence of a catalyst, for example, titanium silicate. For the synthesis of Formula (IB), the most common preparation can be completed by reacting the oxime Formula (IA) with an alkylating agent, for example, but not limited to, an alkyl halide, in the presence of a strong base, such as sodium hydride or potassium hydride. Formula (IC) can also be prepared by reacting Formula (IA) with an acyl halide in the presence of a base such as triethylamine, or with an anhydride, for example, but not limited to, acetic anhydride, in the presence of an acid catalyst, for example, perchloric acid, p-toluene sulfonic acid or sulfuric acid. In the scheme, R₁ is a hydrogen atom or sec-butyl group, R₂ is a hydrogen atom or methyl group, R₃ is a hydrogen atom or C5 branched alkyl group, R₄ is a hydrogen atom or methyl group, R₆ is a C1-C5 linear or branched alkyl group, C2-C5 linear or branched alkenyl group, or C3-C6 substituted or unsubstituted cycloalkyl group, and R₇ is a C1-C5 linear or branched alkyl group, C2-C5 linear or branched alkenyl group, or C1-C5 linear or branched alcohol group.

### 4. Compositions

In certain embodiments, the presently disclosed subject matter can provide a flavor or fragrance composition.

In certain embodiments, the presently disclosed subject matter provides a flavor or fragrance composition comprising a compound Formula (I),

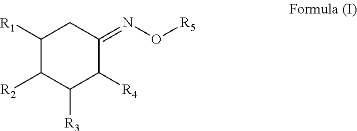

wherein
R₁ is a hydrogen atom or methyl group,
R₂ is a hydrogen atom or C5 branched alkyl group,
R₃ is a hydrogen atom or methyl group,
R₄ is a hydrogen atom or sec-butyl group,
R₅ is a hydrogen atom, C1-C5 linear or branched alkyl group, C2-C5 linear or branched alkenyl group, C1-C5 linear or branched alcohol group, or C(O)R₆, and
R₆ is a C1-C5 linear or branched alkyl group or C3-C6 substituted or unsubstituted cycloalkyl group;

Non-limiting examples of suitable C5 branched alkyl groups at R₂ include, but are not limited to, a pentyl group, 2-methylbutan-2-yl group, 2,2-dimethylpropyl group, 3-methylbutyl group, pentan-2-yl group, pentan-3-yl group, 3-methylbutan-2-yl group, 2-methylbutyl group.

Non-limiting examples of suitable C1-C5 linear or branched alkyl groups at R₅ and/or R₆ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₅ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl groups at R₆ include, but are not limited to, a cyclopropyl group, a cyclopentyl group, and a cyclohexyl group.

Non-limiting examples of suitable C1-C5 linear or branched alcohol groups at R₅ include, but are not limited to, a methyl alcohol, an ethyl alcohol, an n-propyl alcohol, an isopropyl alcohol, and an n-butyl alcohol.

In certain embodiments, R₁ is a hydrogen atom or methyl group.

In certain embodiments, R₂ is a hydrogen atom or a 2-methylbutan-2-yl group.

In certain embodiments, R₃ is a hydrogen atom or methyl group.

In certain embodiments, R₄ is a hydrogen atom or sec-butyl group.

In certain embodiments, R₅ is a hydrogen atom, a methyl group, an ethyl group, an n-butyl group, an allyl group, a 3-methyl-2-butenyl group, an n-propyl alcohol group, or a C(O)R₆ group.

In certain embodiments, R₆ is a methyl group, an ethyl group, an isopropyl group, a cyclopropyl group, or a cyclohexyl group.

In certain embodiments, the compound of Formula (I) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, 2-(sec-butyl)cyclohexan-1-one O-isobutyryl oxime, 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, 3,5-dimethylcyclohexan-1-one O-cyclohexanecarbonyl oxime, 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl oxime, and 4-(tert-pentyl)cyclohexan-1-one O-cyclohexanecarbonyl oxime.

In certain embodiments, the compounds include constitutional isomers, enantiomers, stereoisomers, and racemic mixtures of said compounds listed herein.

In certain embodiments, the present disclosure also provides methods of modifying the odor properties of a fragrance composition by adding to the fragrance composition an olfactory effective quantity of a fragrance compound of Formula (I).

In certain embodiments, the presently disclosed subject matter provides a flavor or fragrance composition comprising a compound Formula (II),

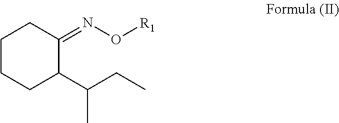

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.

Non-limiting examples of suitable C1-C5 linear or branched alkyl groups at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C1-C5 linear or branched alcohol groups at R₁ include, but are not limited to, a methyl alcohol, an ethyl alcohol, an n-propyl alcohol, an isopropyl alcohol, and an n-butyl alcohol.

In certain embodiments, R₁ is a hydrogen atom, a methyl group, an ethyl group, an n-butyl group, an allyl group, a 3-methyl-2-butenyl group, an n-propyl alcohol group, or a C(O)R₂ group.

In certain embodiments, R₂ is a methyl group, an ethyl group, or an isopropyl group.

In certain embodiments, the compound of Formula (II) is selected from the group consisting of 2-(sec-butyl)cyclohexan-1-one oxime, 2-(sec-butyl)cyclohexan-1-one O-methyl oxime, 2-(sec-butyl)cyclohexan-1-one O-allyl oxime, 2-(sec-butyl)cyclohexan-1-one O-ethyl oxime, 2-(sec-butyl)cyclohexan-1-one 0-(3-methylbut-2-en-1-yl) oxime, 2-(sec-butyl)cyclohexan-1-one O-butyl oxime, 2-(sec-butyl)cyclohexan-1-one 0-(3-hydroxypropyl) oxime, 2-(sec-butyl)cyclohexan-1-one O-acetyl oxime, 2-(sec-butyl)cyclohexan-1-one O-propionyl oxime, and 2-(sec-butyl)cyclohexan-1-one O-isobutyryl oxime.

In certain embodiments, the compounds include constitutional isomers, enantiomers, stereoisomers, and racemic mixtures of said compounds listed herein.

In certain embodiments, the present disclosure also provides methods of modifying the odor properties of a fragrance composition by adding to the fragrance composition an olfactory effective quantity of a fragrance compound of Formula (II).

In certain embodiments, the presently disclosed subject matter provides a flavor or fragrance composition comprising a compound Formula (III),

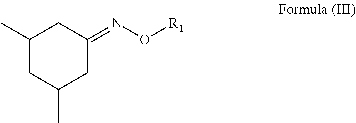

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

Non-limiting examples of suitable C1-C5 linear or branched alkyl group at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl group at R₂ include, but are not limited to, a cyclopropyl group, a cyclopentyl group, and a cyclohexyl group.

Non-limiting examples of suitable C1-C5 linear or branched alcohol group at R₁ include, but are not limited to, a methyl alcohol, an ethyl alcohol, an n-propyl alcohol, an isopropyl alcohol, and an n-butyl alcohol.

In certain embodiments, R₁ is a hydrogen atom, an ethyl group, an allyl group, an n-propyl alcohol group, or a C(O)R₂ group.

In certain embodiments, R₂ is a methyl group, an ethyl group, an isopropyl group, a cyclopropyl group, or a cyclohexyl group.

In certain embodiments, the compound of Formula (III) is selected from the group consisting of 3,5-dimethylcyclohexan-1-one oxime, 3,5-dimethylcyclohexan-1-one O-allyl oxime, 3,5-dimethylcyclohexan-1-one O-ethyl oxime, 3,5-dimethylcyclohexan-1-one 0-(3-hydroxypropyl) oxime, 3,5-dimethylcyclohexan-1-one O-acetyl oxime, 3,5-dimethylcyclohexan-1-one O-propionyl oxime, 3,5-dimethylcyclohexan-1-one O-isobutyryl oxime, 3,5-dimethylcyclohexan-1-one O-cyclopropanecarbonyl oxime, and 3,5-dimethylcyclohexan-1-one O-cyclohexanecarbonyl oxime.

In certain embodiments, the compounds include constitutional isomers, enantiomers, stereoisomers, and racemic mixtures of said compounds listed herein.

In certain embodiments, the present disclosure also provides methods of modifying the odor properties of a fragrance composition by adding to the fragrance composition an olfactory effective quantity of a fragrance compound of Formula (III).

In certain embodiments, the presently disclosed subject matter provides a flavor or fragrance composition comprising a compound Formula (IV),

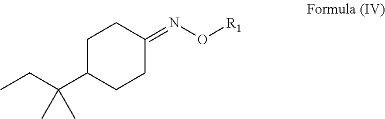

wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group or C3-C6 substituted or unsubstituted cycloalkyl group.

Non-limiting examples of suitable C1-C5 linear or branched alkyl group at R₁ and/or R₂ include, but are not limited to, a methyl group, an ethyl group, an n-propyl group, an isopropyl group, an n-butyl group, a 2-butyl group, an isobutyl group, a tert-butyl group, n-pentyl group, a 2-pentyl group, a 3-pentyl group, a 2-methylbutyl group, a 3-methylbutyl group, a 2-methylbutan-3-yl group, and a 2,2-dimethylpropyl group.

Non-limiting examples of suitable C2-C5 linear or branched alkenyl groups at R₁ include, but are not limited to, a vinyl group, a 1-propenyl group, a 2-propenyl group, 2-methyl-1-propenyl group, an allyl group, and a 3-methyl-2-butenyl group.

Non-limiting examples of suitable C3-C6 substituted or unsubstituted cycloalkyl group at R₂ include, but are not limited to, a cyclopropyl group, a cyclopentyl group, and a cyclohexyl group.

Non-limiting examples of suitable C1-C5 linear or branched alcohol group at R₁ include, but are not limited to, a methyl alcohol, an ethyl alcohol, an n-propyl alcohol, an isopropyl alcohol, and an n-butyl alcohol.

In certain embodiments, R₁ is a hydrogen atom, a methyl group, an allyl group, or a C(O)R₂ group.

In certain embodiments, R₂ is a methyl group, an ethyl group, an isopropyl group, a cyclopropyl group, or a cyclohexyl group.

In certain embodiments, the compound of Formula (IV) is selected from the group consisting of 4-(tert-pentyl)cyclohexan-1-one oxime, 4-(tert-pentyl)cyclohexan-1-one O-methyl oxime, 4-(tert-pentyl)cyclohexan-1-one O-allyl oxime, 4-(tert-pentyl)cyclohexan-1-one O-acetyl oxime, 4-(tert-pentyl)cyclohexan-1-one O-propionyl oxime, 4-(tert-pentyl)cyclohexan-1-one O-isobutyryl oxime, 4-(tert-pentyl)cyclohexan-1-one O-cyclopropanecarbonyl, and 4-(tert-pentyl)cyclohexan-1-one O-cyclohexanecarbonyl oxime.

In certain embodiments, the compounds include constitutional isomers, enantiomers, stereoisomers, and racemic mixtures of said compounds listed herein.

In certain embodiments, the present disclosure also provides methods of modifying the odor properties of a fragrance composition by adding to the fragrance composition an olfactory effective quantity of a fragrance compound of Formula (IV).

### 4.1. Fragrance Compositions

In certain embodiments, any one of the above described compounds can be provided in a fragrance composition. Certain embodiments of the presently disclosed subject matter provide a method to modify, enhance or improve the odor properties of a fragrance composition by adding to the composition an effective quantity of one or more of the compounds of Formula (I), Formula (II), Formula (III), or Formula (IV).

The compounds of Formula (I), Formula (II), Formula (III), or Formula (IV) are particularly valuable as being capable of imparting specific beneficial notes to a fragrance composition.

For fragrance applications, concentrations of the compounds of Formula (I), Formula (II), Formula (III), or Formula (IV) are based on the total weight of the composition into which the fragrance compound is incorporated. For fragrance applications, typical concentrations of the presently disclosed compounds range from about 0.001% to about 20% by weight, preferably from about 0.01% to about 10% by weight, more preferably from about 0.1% to about 5%, more preferably from about 1% to about 3%, based on the total weight of the composition into which the fragrance compound is incorporated. Those skilled in the art are able to employ the desired level of the compounds of the disclosed subject matter to provide the desired fragrance and intensity.

The compounds of the presently disclosed subject matter can be combined with one or more fragrance accords or fragrance compounds from various fragrance categories, including but not limited to one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof in order to create more complex fragrance composition.

Non-limiting examples of suitable aldehydic compounds include 2,6,10-trimethyl-9-undecenal, aldehyde C-6, aldehyde C-7, aldehyde C-8, aldehyde C-9, aldehyde C-10, aldehyde C-11, aldehyde C-12 lauric, aldehyde C-12 MNA, 9-undecenal, octahydro-8,8-dimethylnaphthalene-2-carbaldehyde, octahydro-5,5-dimethylnaphthalene-2-carbaldehyde, trans-2-decenal, trans-4-decenal, cis-4-decenal, 9-decenal, isohexenyl cyclohexenyl carboxaldehyde, 1,2,3,4,5,6,7,8-Octahydro-8,8-dimethyl-2-naphthaldehyde, trans-2-decenal, undecylenic aldehyde, 1-formyl-1-methyl-4-(4-methyl-pentyl)-3-cyclohexene, and combinations thereof.

Non-limiting examples of an animalic compound are 5-cyclohexadecen-1-one, omega-6-Hexadecenlactone, octahydro-2,5,5-trimethyl-2-naphthol, 2-methyl-4-(camphenyl-8)-cyclohexanone, civet, p-cresol, skatole, and combinations thereof.

Non-limiting examples of a balsamic compound are benzyl salicylate, cyclohexyl salicylate, phenethyl salicylate, styrax oil, and combinations thereof.

Non-limiting examples of a citrus compound are delta-3-carene, citral, citronellal, myrcenol, dihydromyrcenol, nonyl acetate, nootkatone, sinensal, bergamot oil, grapefruit oil, lemon oil, lime oil, tridecene-2-nitrile, citral diethyl acetal, d-limonene, terpinolene, and combinations thereof.

Non-limiting examples of a floral compound are acetanisole, alpha amyl cinnamaldehyde, anisyl acetate, anisic aldehyde, benzyl acetate, p-tert-butyldihydrocinnamaldehyde, p-isobutyl-alpha-methyl hydrocinnamaldehyde, butyl acetate, 1-citrol, linalool, geraniol, dl-citronellol, linalyl acetate, cyclamen aldehyde, cyclohexyl lactone, delta-damascone, 9-decen-1-ol, dimethyl benzyl carbinol, farnesal, 1-dihydrofarnesal, farnesol, isopropylphenylbutanal, alpha,alpha-dimethyl-p-ethylphenylpropanal, 2-isobutyl-4-methyltetrahydro-2H-pyran-4-ol, geranyl acetate, piperonal, alpha-methyl-1,3-benzodioxole-5-propionaldehyde, hexyl cinnamaldehyde, indole, alpha-ionone, beta-ionone, alpha-isomethyl ionone, hexyl salicylate, isopropoxy ethyl salicylate, methyl dihydrojasmonate, cis-jasmone, 4-(4-hydroxy-4-methylpentyl)-3-cyclohexene-1-carboxaldehyde, 2,4,6-trimethyl-4-phenyl-1,3-dioxane, 2,4-dimethyl-4,4a,5,9b-tetrahydroindeno[1,2-d]-1,3-dioxin, methyl dihydrojasmonate, methoxymelonal, nerol, nerolione, neryl acetate, 2-pentyl cyclopentanone, alpha-cyclohexylidene benzeneacetonitrile, 3-methyl-5-phenylpentanol, phenylacetaldehyde, phenyl ethyl alcohol, rose oxide, rosephenone, 2,2,5-trimethyl-5-pentylcyclopentanone, yara yara, geranium oil, rose oil, ylang oil, 1-dihydro farnesol, 1-(2,6,6-trimethylcyclohex-2-en-1-yl)hepta-1,6-dien-3-one, diphenyl ether and combinations thereof.

Non-limiting examples of a fruity compound are aldehyde C-16, allyl caproate, allyl cyclohexyl propionate, allyl heptanoate, amyl acetate, benzaldehyde, cassis oil, 1-citronellyl acetate, 1-citronellyl nitrile, acetoxydihydrodicyclopentadiene, 3a,4,5,6,7,7a-hexahydro-4,7-methano-1H-indenyl propionate, damascenone, decyl acetate, gamma-decalactone, diethyl malonate, dimethyl benzyl carbinol acetate, dimethyl phenyl ethyl carbinol, dimethyl sulfide, γ-dodecalactone, ethyl acetate, ethyl butyrate, ethyl caproate, ethyl decadienoate, ethyl-2-methylbutyrate, ethyl acetoacetate, ethyl propionate, ethyl (3a.alpha.,4.beta.,7.beta.,7a.alpha.)-octahydro-4,7-methano-3aH-indene-3a-carboxylate, hexyl acetate, hexyl isobutyrate, isoamyl acetate, jasmolactone, ethyl 2-methylpentanoate, melonal, methyl heptyl ketone, gamma-nonalactone, octyl acetate, gamma-octalactone, phenyl ethyl isobutyrate, raspberry ketone, p-mentha-8-thiol-3-one, tolyl aldehyde, γ-undecalactone, 3,5,5-trimethylhexyl acetate, methyl dioxolane, ethyl decadienoate, 2-tert-butylcyclohexyl acetate, 3-methyl-2-butenyl acetate, alpha,alpha-dimethylphenethyl butyrate, 2-isopropyl-4-methylthiazole, 4-tert-butylcyclohexyl acetate, and combinations thereof.

Non-limiting examples of a gourmand compound are caprylic acid, 2-ethyl-4-hydroxy-5-methyl-3(2H)-furanone, ethyl vanillin, methylheptenone, 4-hydroxy-2,5-dimethyl-3(2H)-furanone, guaiacol, 5-ethyl-3-hydroxy-4-methyl-2(5H)-furanone, 2-acetyl pyrazine, 2,5-dimethyl pyrazine, coumarin, and combinations thereof.

Non-limiting examples of a green compound are allyl amyl glycolate, 1-(5,5-dimethyl-1-cyclohexen-1-yl)pent-4-en-1-one, 1,3,5-undecatriene, trans-2-hexenal, cis-3-hexenol, hexen-1-ol, cis-3-hexenyl acetate, cis-3-hexenyl butyrate, cis-3-hexenyl formate, cis-3-hexenyl salicylate, cis-3-hexenyl methyl carbonate, 2-methoxy-2-methylheptane, methyl octine carbonate, methyl 2-nonenoate, 2,6-nonadienal, cis-2-methyl-4-propyl-1,3-oxathiane, methoxy dicyclopentadiene carboxaldehyde, 5-methyl-3-heptanone oxime, styrallyl acetate, 2,4-dimethyl-3-cyclohexen-1-carboxaldehyde, 4-methyl-3-decen-5-ol, 2,6-nonadienenitrile, violet leaf extract, allyl (cyclohexyloxy)acetate, tricyclodecenyl propionate, and combinations thereof.

Non-limiting examples of a herbaceous and/or herbal compound are alpha-terpineol, tetrahydrolinalool, 3-methyl-2,4-nonedione, 2-methyl-3-(p-methoxyphenyl)-propanal, carvacrol, clary sage natural oil, 2,6-dimethyl-2-heptanol, natural basil oil, natural cedar leaf oil, spearmint oil, cornmint oil and combinations thereof.

Non-limiting examples of a marine compound are 7-methyl-2H-benzo-1,5-dioxepin-3(4H)-one, alpha,alpha-dimethyl-p-ethylphenylpropanal, (4-tert-butylphenyl)acetonitrile, 4-(4,8-dimethylnona-3,7-dienyl)pyridine, ultrazure, and combinations thereof.

Non-limiting examples of a mossy compound are isobutyl quinolone, isopropyl quinolone, methyl atrarate, and combinations thereof.

Non-limiting examples of a musk compound are ethylene brassylate, omega-pentadecalactone, 2-(1-(3′,3′-dimethyl-1′-cyclohexyl)ethoxy)-2-methyl propyl propanoate, (1′R)-3-methyl-5-(2,2,3-trimethylcyclopentan-1-yl)-2-pentanone, 3-methylcyclopentadecenone, 5-acetyl-1,1,2,3,3,6-hexamethylindan, 1,3,4,6,7,8-hexahydro-4,6,6,7,8,8-hexamethylcyclopenta-gamma-2-benzopyran and combinations thereof.

Non-limiting examples of a piney compound are 1-borneol, 1-bornyl acetate, camphene, dihydroterpineol, β-pinene, Camphor gum powder, and combinations thereof.

Non-limiting example of a powdery compound is, methyl octalactone, heliotropin and a combination thereof.

Non-limiting examples of a spicy compound are delta-caryophyllene, cinnamyl alcohol, cinnamaldehyde, cuminaldehyde, eugenol, isoeugenol, p-mentha-1,8-dien-7-al, cardamom oil, clove oil, ginger extract, black pepper extract and combinations thereof.

Non-limiting examples of a woody and/or amber compound are 1-(2-tert-butyl cyclohexyloxy)-2-butanol, decahydro-2,2,6,6,7,8,8-heptamethyl 2H-indeno[4,5b]furan, (4aR,5R,7aS,9R)-Octahydro-2,2,5,8,8,9a-hexamethyl-4H-4a,9-methanozuleno[5,6-d]-1,3-dioxole, 2-ethyl-4-(2,2,3-trimethyl-3-cyclopenten-1-yl)-2-buten-1-ol, formaldehyde cyclododecyl ethyl acetal, 6,7-dihydro-1,1,2,3,3-pentamethyl-4(5H)-indanone, cedrol methyl ether, exo-2-[(1,7,7-trimethylbicyclo[2.2.1]hept-2-yl)oxy]ethanol, 3-methyl-5-(2,2,3-trimethyl-3-cyclopenten-1-yl)pent-4-en-2-ol, 2-methyl-4-(2,2,3-trimethyl-3-cyclopenten-1-yl)-2-buten-1-ol, hinokitiol, DH-ionone beta, 1-methyl-2-[(1,2,2-trimethylbicyclo[3.1.0]hex-3-yl)methyl]-cyclopropanemethanol, 2-(2,4-dimethyl-3-cyclohexene-1-yl)-5-methyl-5-(1-methylpropyl)-1,3-dioxane, 2,4-dimethyl-2-(5,6,7,8-tetrahydro-5,5,8,8-tetramethyl-2-naphthalenyl)-1,3-dioxolane, 1-(2,3,8,8-tetramethyl-1,3,4,5,6,7-hexahydronaphthalen-2-yl)ethanone, patchouli oil, sandalwood oil, 4-tert-butylcyclohexanol, acetyl cedrene, 1-(1,2,3,4,5,6,7,8-Octahydro-2,3,8,8-tetramethyl-2-naphthalenyl)ethenone, 1,3-dimethyl-3-phenylbutyl acetate, and combinations thereof.

Those skilled in the art will recognize that fragrance compounds not listed in the examples above can be selected on the basis of his or her knowledge of fragrance compounds from various fragrance categories or by referencing the odor properties of fragrance compounds as listed in databases such as, the IFRA FRAGRANCE INGREDIENT GLOSSARY (ifrafragrance.org/priorities/ingredients/glossary).

The compounds of the disclosed subject matter provide unique and beneficial odor properties. For example, 2-(sec-butyl)cyclohexan-1-one oxime provides green, grassy, cassis, powerful, galbanum, fig leaf, vegetal, clean, earthy floral odor properties. For example, 2-(sec-butyl)cyclohexan-1-one O-methyl oxime provides fruity, berry, black currant, jackfruit, pineapple, exotic, dirty, slightly animalic, natural leafy green odor properties.

Such compositions can contain or consist of at least one fragrance compound selected from a group consisting of a fragrance carrier and a fragrance base.

Fragrance carriers can be a liquid or a solid and typically do not significantly alter the olfactory properties of the fragrance compounds. Some non-limiting examples of fragrance carriers include an emulsifying system, encapsulating materials (e.g., melamine-formaldehyde resin and urea-formaldehyde resin), natural or modified starches, polymers(e.g., polyurea, polyamide, co-polymers of acrylic acid, co-polymers of methacrylic acid, and their esters), gums (e.g., tamarind seed gum, guar gum, and locust bean gum, gum arabic, and gum karaya), pectins, gelatinous or porous cellular materials, waxes, and solvents which are typically employed in fragrance applications.

Fragrance base refers to any composition comprising at least one fragrance compound. In general, these fragrance compounds belong to chemical classes such as, but not limited to: alcohols, aldehydes, ketones, esters, ethers, acetals, oximes, acetates, nitriles, terpenes, saturated and unsaturated hydrocarbons, and essential oils of natural or synthetic origins.

The fragrance compositions of the presently disclosed subject matter can be in the form of a simple mixture of the various fragrance compounds and solvents, or also in the form of a biphasic system such as an emulsion or microemulsion. Such systems are well-known to persons skilled in the art.

Nonlimiting examples of such solvents used in perfumery are known in the art and include but are not limited to: dipropyleneglycol, diethyl phthalate, isopropyl myristate, benzyl benzoate, 2-(2ethoxy)-1-ethanol, ethyl citrate, ethanol, water/ethanol mixtures, isoparaffins such as those known under the trademark Isopar© (ExxonMobil Chemicals, Houston, TX), and glycol ethers and glycol ether esters such as those known under the trademark Dowanol©(Dow Chemical Company, Midland MI).

### 4.2. Use of Fragrance Compositions in Consumer Products

Suitable consumer products that can include a fragrance compound or fragrance composition of the presently disclosed subject matter include, but are not limited to: 1) air care products; 2) fine fragrances; 3) personal care products; 4) cosmetics, e.g., lotions, creams and ointments for the skin, color cosmetics; and 5) fabric and home care products, e.g., laundry detergents, fabric softeners and conditioners, rinse additives; dish detergents, household and institutional cleansers/cleaning agents, 6) pharmaceutical and over-the-counter (OTC) products,; and 7) insect repellents or insecticides; and 8) pet care products, e.g., pet deodorizers, pet shampoo, pet conditioner, pet litter. Depending on the solvents that can be present in some end products, it can be necessary to protect the compounds from premature degradation, for example by encapsulation or with a stabilizer, or other methods well-known to those of ordinary skill in the art.

Non-limiting examples of air care include candles, air fresheners and the like.

Non-limiting examples of fine fragrance products include extrait de parfum, eau de perfume, eau de toilet, eau de cologne, baby cologne and the like.

Non-limiting examples of personal care products include antiperspirants, deodorants, shave lotions, cream, foam and gels, bath soaps, perfumed soaps, transparent soaps, synthetic soaps, body cleansers, hand soaps, bath salts, bath tablets, bath liquids, foam and bubble baths, bath oils, bath perfumes, bath capsules, milk baths, bath gels, bath cubes, body wipes, infant and toddler care wipes and the like. Personal care products can also include family care products (e.g., wet or dry bath tissue, facial tissue, disposable handkerchiefs, disposable towels, and/or wipes, towels, toilet paper, tissue paper, wet towels, etc.); feminine care products (e.g., catamenial pads, incontinence pads, interlabial pads, panty liners, pessaries, sanitary napkins, tampons and tampon applicators, and/or wipes, etc.).

Non-limiting examples of cosmetics include skin-care cosmetics, face washing creams, varnishing creams, cleansing creams, cold creams, massage creams and oils, milky lotions, skin toning lotion, makeup remover, makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, suntan products, sunscreen products, and the like. Personal care product can include hair care products. Non-limiting examples of hair care products include shampoo, hair conditioners, hair treatment, hair pack, pomades, rinses, brilliantines, setting lotions, hair sticks, hair solids, hair oils, hair treatments, hair creams, hair tonics, hair liquids, hair sprays, hair restorers, hair dyes, permanent wave lotions, and the like.

Non-limiting examples of fabric and home care products can include wood polisher, wood cleaner, car care products; e.g., car cleaner, car wax, dish detergent, dishwasher cleaner, hard surface cleaner, hard surface cleaning wipes, disinfectant wipes, disinfectant spray, dishwasher cleaner, dish detergent, upholstery cleaners, upholstery deodorizers, carpet cleaner, carpet deodorizer and the like. Fabric and home care products can include laundry care products. Non-limiting examples of laundry care products include laundry detergents (e.g., heavy duty detergents for clothes, light duty detergents for clothes, liquid detergents, laundering soaps, compact detergents, powder soaps), softening finishing agents, softeners, bleaching agent, stain remover, fabric deodorizer and the like.

Non-limiting examples of pharmaceutical and over-the-counter (OTC) products include medicated soaps, medicated shampoos, medicated skin care products, medicated patch and the like.

Non-limiting examples of insect repellents or insecticides include spray type, aerosol type, sheet type, vapor type, liquid type, oil type, water type and cream type of insect repellents or insecticides.

Non-limiting examples of pet care products include pet deodorizers, pet shampoo, pet conditioner, pet litter, pet soap, pet cream, and other topically applied products.

The compositions of the presently disclosed subject matter can be included in many additional consumer products.

### 4.3. Flavor Compositions

In certain embodiments, any one of the above-described compounds can be provided in a flavor composition. Certain embodiments of the presently disclosed subject matter provide a method to modify, enhance, or improve the taste properties of a flavor composition by adding to the composition an effective quantity of one or more of the presently disclosed compounds of Formula (I), Formula (II), Formula (III), or Formula (IV).

Those skilled in the art are able to employ the desired level of said compounds to provide the desired flavor and intensity. Much higher concentrations can be employed when the compounds are used in concentrated flavors and flavor compositions. For flavor applications, concentrations of compounds of Formula (I), Formula (II), Formula (III), or Formula (IV) are based on the total weight of the composition into which the compound is incorporated. For flavor applications, typical concentrations of the compounds of Formula (I), Formula (II), Formula (III), or Formula (IV) range from about 0.0005% to about 20% by weight, from about 0.0005% to about 10% by weight, from about 10% to about 20% by weight, from about 0.001% to about 20% by weight, preferably from about 0.01% to about 10% by weight, more preferably from about 0.1% to about 5% by weight, and more preferably from about to 1% to about 3% by weight based on the total weight of the composition into which the compound is incorporated.

As used herein, organoleptic effective quantity is defined as the amount of said compound in a flavor composition in which the individual component will contribute its characteristic flavor properties. However, the organoleptic effect or taste profile of the flavor composition will be the sum of the effects of all flavor compounds present. Therefore, the compounds embodied in the presently disclosed subject matter can be employed in more complex flavor composition comprising one or more additional flavor compounds, or additional flavor compositions, to modify the overall taste characteristics of the flavor composition via their own organoleptic properties or through enhancing or complimenting the contributions of the additional flavor compounds present within the said composition. Such additional flavor compounds or flavor compositions include, for example, natural or synthetic flavors, i.e., fruit flavors (e.g., lemon, lime, orange, grapefruit; cherry, strawberry, raspberry, cranberry, apple, grape, pineapple, banana, tomato); natural or synthetic botanical flavors (e.g., tea flavors, coffee flavors, hazelnut, almond, pecan or other nut flavors, vanilla flavors), and flavor compositions with complex flavor profiles (e.g., cola flavors or imagined flavors, such as “birthday cake” or “ice cream sundae”). The quantity of the presently disclosed compound in such more complex flavor compositions will vary widely depending on the presence of additional flavor compounds present, their relative amounts, the desired taste profile, and the nature of the consumer product in which the flavor composition will be utilized.

The flavor carrier can be a liquid or a solid, and typically does not significantly alter the olfactory or organoleptic properties of the flavor compounds, respectively. Some non-limiting examples of flavor carriers include an emulsifying system, encapsulating materials, natural or modified starches, polymers, pectins, proteins, polysaccharides, gums and solvents which are typically employed in flavor applications.

The flavor compositions according to the disclosed subject matter can be in the form of a simple mixture of the various flavor compounds, and solvents, or also in the form of a biphasic system such as an emulsion or microemulsion. Such systems are well-known to persons skilled in the art.

The flavor composition can include one or more of the following materials: solvents, support materials, diluents, carriers, preservatives, flavor enhancers, antioxidants, color retention agents, dyes or lakes, sequestrants, emulsifiers, stabilizers, acids, bases, and/or anticaking agents. The one or more noted materials can be selected from the following non-limiting examples of materials: purified water, glycerol, medium-chain triglycerides (MCTs), propylene glycol, triacetin, triethyl citrate, benzyl alcohol, benzyl benzoate, ethanol, vegetable oils, terpene, sulfites, sodium nitrite, propionic acid, sorbic acid, benzoic acid, disodium ethylenediaminetetraacetic acid (EDTA), alcohols, esters, aldehydes, ketones, lactones, phenols, monosodium glutamate (MSG), monopotassium glutamate, calcium diglutamate (CDG), guanosine monophosphate disodium guanylate, sodium guanylate, inosinic acid and its salts, L-leucine, ascorbic acid, sodium ascorbate, fatty acid esters of ascorbic acid, tocopherols, alpha-tocopherol, gamma-tocopherol, delta-tocopherol, propyl gallate, octyl gallate, erythorbic acid, sodium erythorbate, dodecyl gallate, tertiary-butyl hydroquinone (TBHQ), butylated hydroxyanisole (BHA), butylated hydroxytoluene (BHT), citric acid, 4-hexylresorcinol, lecithins, monoglyceride, acetylated monoglyceride, lactylated monoglyceride, sugar ester, sorbitan ester, polyglycerol ester, calcium stearoyl di laciate, calcium silicate, magnesium carbonate, and sodium aluminosilicate

### 4.4. Use of Flavor Compositions in Consumer Products

The presently disclosed compounds of Formula (I), Formula (II), Formula (III), or Formula (IV) or flavor compositions of the presently disclosed subject matter can be used in consumer products such as, but are not limited to: 1) foods or beverages, 2) oral care products, 3) animal feed and pet foods, 4) pharmaceuticals (including over-the-counter medications), and 5) sexual health care products. Depending on the solvents that can be present in some end products, it can be necessary to protect the compounds from premature degradation, for example by encapsulation or with a stabilizer, or other methods well-known to those of ordinary skill in the art.

Non-limiting examples of food and beverages can include, carbonated or non-carbonated fruit beverages, carbonated cola drinks, wine coolers, cordials, flavored water, powders for drinks (e.g., powdered sports or “hydrating” drinks), fruit based “smoothie” drinks, vegetable based “smoothie” drinks, milk-based drinks, hard candy, soft candy, taffy, chocolates, sugarless candies, chewing gum, bubble gum, snack foods, desserts(e.g., jelly, pudding), confectionery (e.g., cookies, cakes, condiments), spices and seasonings, dry cereal, oatmeal, granola bars, condiments and preserves, soups, alcoholic beverages, non-alcoholic drink mixes (e.g., margarita or piña colada concentrates), energy beverages, juices, teas, coffees, salsa, frozen confectioneries (e.g., ice creams, sherbets, and ice-lollies), gelatin candies, pectin candies, starch candies, and lozenges.

Non-limiting examples of oral care products can include film strips for halitosis, chewable tablets, breath mints, oral wash, tooth gel, toothpastes, and mouth rinses.

Non-limiting examples of animal feed and pet foods can include wet type pet food, dry type pet food, pet treats, and oral care treats for pets.

Non-limiting examples of pharmaceutical product can include cough drops, throat lozenges, throat sprays, nicotine gums, decongestants, oral analgesics, indigestion preparations, and antacids.

Non-limiting examples of oral care products can include mouthwash, toothpaste, floss, and dental pick.

Non-limiting examples of sexual health care products can include lubricants and condoms.

The flavor compositions according to the disclosed subject matter can be in the form of a simple mixture of flavor compounds or in an encapsulated form, e.g., a flavoring composition entrapped into a solid matrix that can comprise wall-forming and plasticizing materials such as mono-, di-, or trisaccharides, natural or modified starches, hydrocolloids, cellulose derivatives, polyvinyl acetates, polyvinylalcohols, proteins, or pectins. Examples of particularly useful matrix materials include, for example, sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum accacia, tragacanth, alginates, methyl cellulose, carboxymethyl cellulose, hydroxyethyl cellulose, hydroxypropylmethyl cellulose, derivatives, gelatin, agar, alginate, and mixtures thereof. Encapsulation is well-known to persons skilled in the art, and can be performed, for instance, using techniques such as spray-drying, agglomeration or extrusion, or coating encapsulation, including coacervation and complex coacervation techniques.

### EXAMPLES

The present application is further described by means of the examples, presented below, wherein the abbreviations have the usual meaning in the art.

The use of such examples is illustrative only and does not limit the scope and meaning of the disclosed subject matter or of any exemplified term. Likewise, the disclosed subject matter is not limited to any particular preferred embodiments described herein. Indeed, many modifications and variations of the disclosed subject matter are apparent to those skilled in the art upon reading this specification. The disclosed subject matter is therefore to be limited only by the terms of the appended claims along with the full scope of equivalents to which the claims are entitled.

The following Examples are provided as specific embodiments of the presently disclosed subject matter, wherein the abbreviations have the usual meaning in the art. The temperatures are indicated in degrees centigrade (° C.); the NMR spectral data were recorded in CDCl₃ with a JEOL 400 MHz machine for ¹H and ¹³C, the chemical displacements are indicated in ppm with respect to TMS as the standard.

All solid compounds synthesized in examples 1-27 were evaluated as a 10% dilution in TEC (triethyl citrate).

### Example 1: Synthesis of 2-(sec-butyl)cyclohexan-1-one oxime

To a one neck round bottom flask, was added the 11.0 g (71 mmol) of 2-(sec-butyl)cyclohexan-1-one, ethanol (125 mL) and 11.28 g (140 mmol) pyridine. The mixture was stirred at room temperature while under nitrogen and then 5.45 g (77 mmol) of hydroxylamine HCl was added portion wise. The reaction was stirred at room temperature until completion. The solvent was removed under reduced pressure and the residue was then diluted with ethyl acetate and washed with water, 1M HCl and brine. The aqueous washes were combined and back extracted with ethyl acetate one time. The organics were combined and dried over MgSO₄, filtered, and concentrated under reduced pressure. The crude material was isolated via automated silica gel chromatography (Biotage Isolera, 6-50% ethyl acetate/hexane) with a final isolation by Kugelrohr distillation (70° C. @0.50 Torr) to afford 9.713 g (80.5%) of 2-(sec-butyl)cyclohexan-1-one oxime as a colorless, viscous oil. Odor characteristics: green, grassy, cassis, powerful, galbanum, fig leaf, vegetal, clean, earthy floral.

1H NMR δ ppm 0.86 (m, 6H) 1.04 (m, 1H) 1.37 (dtd, 1H) 1.55 (m, 4H) 1.74 (m, 3H) 2.04 (m, 1H) 2.20 (td, 1H), 2.80 (m, 1H). 13C NMR δ ppm 10.69, 10.87, 11.48, 11.63, 16.05, 16.10, 16.40, 17.10, 20.53, 20.73, 22.12, 22.31, 22.39, 26.12, 26.26, 26.92, 27.41, 27.52, 27.66, 27.91, 28.63, 28.72, 29.41, 32.24, 32.55, 32.82, 32.99, 37.35, 38.90, 46.61, 46.70, 163.00, 163.13, 163.67, 163.93.

### Example 2: Synthesis of 2-(sec-butyl)cyclohexan-1-one O-methyl oxime

To a one neck round bottom flask, was added 6.5 g (42 mmol) of 2-(sec-butyl)cyclohexan-1-one, ethanol (125 mL) and (85 mmol) pyridine. The mixture was stirred at room temperature while under nitrogen and then 3.88 g (46 mmol) of N-methoxyamine hydrochloride was added portion wise. The reaction was stirred at room temperature until completion. The solvent was removed under reduced pressure and the residue was then diluted with ethyl acetate and washed with water, 1M HCl and brine. The aqueous washes were combined and back extracted with ethyl acetate one time. The organics were combined and dried over MgSO₄, filtered and concentrated under reduced pressure. The crude material was purified via automated silica gel chromatography (Biotage Isolera, 6-50% ethyl acetate/hexane) with a final isolation by Kugelrohr distillation (57° C. @0.90 Torr) to afford 5.929 g (79.1%) of 2-(sec-butyl)cyclohexan-1-one O-methyl oxime as a colorless oil. Odor characteristics: fruity, berry, black currant, jackfruit, pineapple, exotic, dirty, slightly animalic, natural leafy green.

1H NMR δ ppm 0.84 (m, 6H) 1.06 (dsep, 1H) 1.29-2.05 (m, 10H) 2.82 (m, 1H) 3.79 (s, 3H). 13C NMR δ ppm 10.77, 11.51, 16.16, 16.91, 21.76, 21.95, 22.75, 23.01, 26.07, 26.46, 27.29, 28.95, 32.40, 32.83, 46.19, 46.70, 61.05, 162.52, 162.59.

### Example 3: Synthesis of 2-(sec-butyl)cyclohexan-1-one O-allyl oxime

To an oven dried, two neck round bottom flask was added 0.30 g (11.8 mmol) sodium hydride and anhydrous THE (20 mL). The grey suspension was stirred under nitrogen, then cooled to 0° C. in an ice bath. A solution of 1.0 g (5.9 mmol) 2-(sec-butyl)cyclohexan-1-one oxime in anhydrous THE (10 mL) was added dropwise to the suspension at this temperature. After addition, the ice bath was removed to let the mixture warm to room temperature, where it remained for 2 hours. At this point, 0.56 mL (6.5 mmol) allyl bromide was added dropwise. After addition, the reaction was stirred at room temperature until completion. The mixture was cooled to 0° C. in an ice bath, diluted with diethyl ether and quenched with water. The layers were separated, and the aqueous layer was back extracted with diethyl ether (3×). The organics were combined, dried over MgSO₄, filtered, and concentrated under reduced pressure. The crude material was purified via automated silica gel chromatography (Biotage Isolera, 1-10% ethyl acetate/hexanes) to afford 0.591 g (47.7%) of 2-(sec-butyl)cyclohexan-1-one O-allyl oxime as an oil. Odor characteristics: earthy, onion.

1H NMR δ ppm 0.85 (m, 6H) 1.03 (m, 1H) 1.35-1.52 (m, 6H) 1.80 (m, 2H) 2.06 (m, 2H) 2.78-3.15 (m, 1H) 4.49 (m, 2H) 5.16 (m, 1H) 5.27 (m, 1H) 5.97 (m, 1H). 13C NMR δ ppm 10.74, 11.46, 16.17, 16.97, 20.51, 20.74, 21.85, 22.00, 23.00, 23.26, 26.06, 26.46, 28.93, 28.96, 32.47, 32.81, 38.66, 39.65, 46.25, 46.67, 73.90, 74.02, 116.76, 116.80, 134.93, 135.01, 162.87, 163.03.

### Example 4: Synthesis of 2-(sec-butyl)cyclohexan-1-one O-ethyl oxime

To an oven dried, two neck round bottom flask was added 0.41 g (17.1 mmol) sodium hydride and anhydrous THE (20 mL). The grey suspension was stirred under nitrogen, then cooled to 0° C. in an ice bath. A solution of 1.41 g (8.3 mmol) 2-(sec-butyl)cyclohexan-1-one oxime in anhydrous THE (10 mL) was added dropwise to the suspension at this temperature. After addition, the ice bath was removed to let the mixture warm to room temperature, where it remained for 2 hours. At this temperature, 0.76 mL (6.5 mmol) iodoethane was added dropwise. After addition, the reaction continued to stir at room temperature until completion. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 1-10% ethyl acetate/hexanes) to afford 0.257 g (15.2%) of 2-(sec-butyl)cyclohexan-1-one O-ethyl oxime as an oil. Odor characteristics: pickle, oshinko.

1H NMR δ ppm 0.84 (m, 6H) 1.21 (m, 4H) 1.52 (m, 5H) 1.76 (m, 2H) 1.89 (m, 1H) 2.01 (m, 2H) 2.74-3.12 (m, 1H) 4.03 (m, 2H). 13C NMR δ ppm 10.83, 11.50, 14.72, 14.78, 16.19, 16.95, 20.55, 20.78, 21.84, 22.01, 22.90, 23.16, 26.08, 26.46, 28.93, 28.96, 32.45, 32.81, 38.51, 39.44, 46.30, 46.75, 68.47, 68.54, 162.28, 162.45.

### Example 5: Synthesis of 2-(sec-butyl)cyclohexan-1-one O-(3-methylbut-2-en-1-yl) oxime

To an oven dried, two neck round bottom flask was added 0.34 g (14.2 mmol) sodium hydride and anhydrous THE (20 mL). The grey suspension was stirred under nitrogen, then cooled to 0° C. in an ice bath. A solution of 1.2 g (7.1 mmol) 2-(sec-butyl)cyclohexan-1-one oxime in anhydrous THE (10 mL) was added dropwise to the suspension at this temperature. After addition, the ice bath was removed to let the mixture warm to room temperature, where it remained for 2 hours. At this temperature, 0.90 mL (6.5 mmol) 3,3-dimethylallyl bromide was added dropwise. After addition, the reaction continued to stir at room temperature until completion. The mixture was cooled to 0° C. in an ice bath, diluted with diethyl ether and quenched with water. The layers were separated, and the aqueous layer was back extracted with diethyl ether (3×). The organics were combined, dried over MgSO₄, filtered, and concentrated under reduced pressure. The crude material was purified via automated silica gel chromatography (Biotage Isolera, 1-8% ethyl acetate/hexanes) to afford 0.816 g (48.3%) of 2-(sec-butyl)cyclohexan-1-one O-(3-methylbut-2-en-1-yl) oxime as an oil. Odor characteristics: scallion, pickle.

1H NMR δ ppm 0.84 (m, 6H) 1.05 (m, 1H) 1.37 (dt, J=13.62, 6.70 Hz, 1H) 1.53 (m, 5H) 1.73 (m, 7H) 1.88 (m, 1H) 2.01 (m, 2H) 2.78-3.08 (m, 1H) 4.49 (m, 2H) 5.38 (d, J=6.87 Hz, 1H). 13C NMR δ ppm 10.86, 11.50, 16.16, 16.93, 18.29, 20.54, 20.77, 21.89, 22.06, 23.00, 23.25, 25.96, 26.05, 26.46, 28.90, 29.45, 32.48, 32.82, 38.58, 39.47, 46.33, 46.73, 69.99, 120.72, 137.01, 162.48, 162.67.

### Example 6: Synthesis of 2-(sec-butyl)cyclohexan-1-one O-butyl oxime

To an oven dried, two neck round bottom flask was added 0.40 g (16.7 mmol) sodium hydride and anhydrous THE (20 mL). The grey suspension was stirred under nitrogen, then cooled to 0° C. in an ice bath. A solution of 1.41 g (8.3 mmol) 2-(sec-butyl)cyclohexan-1-one oxime in anhydrous THE (10 mL) was added dropwise to the suspension at this temperature. After addition, the ice bath was removed to let the mixture warm to room temperature, where it remained for 2 hours. At this temperature, 1.1 mL (9.2 mmol) iodobutane was added dropwise. After addition, the reaction continued to stir at room temperature until completion. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 1-10% Ethyl Acetate/Hexanes) to afford 1.091 g (57.7%) of 2-(sec-butyl)cyclohexan-1-one O-butyl oxime as an oil. Odor characteristics: potpourri, complex, chemical.

1H NMR δ ppm 0.85 (m, 9H) 1.01 (s, 1H) 1.37 (m, 3H) 1.48 (m, 2H) 1.60 (m, 4H) 1.76 (m, 2H) 1.88 (m, 1H) 2.00 (m, 2H) 2.92 (m, 1H) 3.97 (m, 2H). 13C NMR δ ppm 10.82, 11.49, 14.06, 14.09, 16.20, 16.94, 19.28, 19.37, 20.57, 20.79, 21.84, 22.01, 22.87, 23.13, 26.09, 26.49, 28.93, 28.96, 31.32, 31.37, 32.45, 32.81, 38.50, 39.55, 46.28, 46.74, 72.87, 72.94, 162.27, 162.45.

### Example 7: Synthesis of 2-(sec-butyl)cyclohexan-1-one O-(3-hydroxypropyl) oxime

To an oven dried, two neck round bottom flask was added 0.43 g (17.7 mmol) sodium hydride and anhydrous THE (20 mL). The grey suspension was stirred under nitrogen, then cooled to 0° C. in an ice bath. A solution of 1.50 g (8.9 mmol) 2-(sec-butyl)cyclohexan-1-one oxime in anhydrous THE (10 mL) was added dropwise to the suspension at this temperature. After addition, the ice bath was removed to let the mixture warm to room temperature, where it remained for 2 hours. At this temperature, 0.9 mL (9.8 mmol) 3-bromo-1-propanol was added dropwise. After addition, the reaction continued to stir at room temperature until completion. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 1-15% ethyl acetate/hexanes) to afford 0.365 g (18.2%) of 2-(sec-butyl)cyclohexan-1-one O-(3-hydroxypropyl) oxime as an oil. Odor characteristics: fruity, red apple.

1H NMR δ ppm 0.84 (m, 6H) 1.03 (m, 1H) 1.33 (ddd, J=13.51, 7.56, 3.66 Hz, 1H) 1.53 (m, 4H) 1.79 (m, 5H) 2.02 (m, 2H) 2.53 (br. s., 1H) 2.78 (m, 1H) 3.73 (t, J=5.95 Hz, 2H) 4.15 (t, J=5.72 Hz, 2H). 13C NMR δ ppm 10.81, 11.46, 16.13, 16.99, 21.79, 22.00, 23.00, 23.30, 26.02, 26.47, 27.29, 28.93, 28.96, 32.47, 32.60, 32.81, 46.40, 46.76, 60.65, 70.90, 163.12, 163.26.

### Example 8: Synthesis of 2-(sec-butyl)cyclohexan-1-one O-acetyl oxime

To an oven dried, round bottom flask was added 1.5 g (9 mmol) of 2-(sec-butyl)cyclohexan-1-one oxime and anhydrous DCM (50 mL). Triethylamine (1.53 mL, 11 mmol) was added to the flask and stirred under nitrogen. The flask was cooled to 0° C. where 0.711 mL (10 mmol) of acetyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (90-91° C. @0.2 Torr) to afford 1.204 g (64.3%) of 2-(sec-butyl)cyclohexan-1-one O-acetyl oxime as an oil. Odor characteristics: green, vegetable, bell pepper, mushroom, metallic.

1H NMR δ ppm 0.84 (m, 6H) 1.07 (m, 1H) 1.23-2.03 (m, 9H) 2.12 (d, 2.11) 2.24 (m, 1H) 3.86 (m, 1H). 13C NMR δ ppm 10.52, 10.62, 11.20, 11.39, 15.84, 16.07,16.50, 16.82, 19.82, 19.92, 20.10, 21.44, 24.38, 24.66, 25.90, 26.63, 26.85, 29.00, 29.12, 32.52, 32.96, 40.38, 41.27, 46.07, 46.77, 169.35, 169.41, 171.10, 171.21.

### Example 9: Synthesis of 2-(sec-butyl)cyclohexan-1-one O-propionyl oxime

To an oven dried, round bottom flask was added 1.5 g (9 mmol) of 2-(sec-butyl)cyclohexan-1-one oxime and anhydrous DCM (50 mL). Triethylamine (1.53 mL, 11 mmol) was added to the flask and stirred under nitrogen. The flask was cooled to 0° C. where 0.902 mL (10 mmol) of propionyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (92-95° C. @0.2 Torr) to afford 1.568 g (78.5%) of 2-(sec-butyl)cyclohexan-1-one 0-propionyl oxime as an oil. Odor characteristics: vinegar, fermented, acidic.

1H NMR δ ppm 0.86 (m, 6H) 1.07 (m, 1H) 1.19 (t, 3H) 1.34 (m, 1H) 1.56 (m, 5H) 1.81 (m, 2H) 1.96 (m, 1H) 2.27 (m, 1H) 2.41 (m, 2H) 2.88 (m, 1H). 13C NMR δ ppm 9.18, 9.25, 11.30, 11.43, 15.91, 16.08, 16.55, 16.85, 20.14, 20.35, 21.31, 21.53, 24.38, 24.66, 26.02, 26.58, 28.92, 28.99, 32.99, 40.30, 41.32, 46.14, 46.83, 171.18, 171.29, 171.79 172.59, 172.64.

### Example 10: Synthesis of 2-(sec-butyl)cyclohexan-1-one O-isobutyryl oxime

To an oven dried, round bottom flask was added 1.0 g (6 mmol) of 2-(sec-butyl)cyclohexan-1-one oxime and anhydrous DCM (50 mL). Triethylamine (1.00 mL, 7 mmol) was added to the flask and stirred under nitrogen. The flask was cooled to 0° C. where 0.689 mL (7 mmol) of isobutyryl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure.

The crude product was then isolated by automated silica gel chromatography (Biotage Isolera, 5-40% ethyl acetate/hexane) with final isolation by Kugelrohr distillation (93-96° C. @0.2 Torr) to afford 1.123 g (79.4%) of 2-(sec-butyl)cyclohexan-1-one O-isobutyryl oxime as an oil. Odor characteristics: sour, fruity.

1H NMR δ ppm 0.84 (m, 6H) 1.09 (m, 1H) 1.20 (d, 6H) 1.33 (m, 1H) 1.56 (m, 4H) 1.78 (m, 2H) 1.94 (m, 2H) 2.27 (m, 1H) 2.63 (m, 1H) 2.90 (m, 1H). 13C NMR δ ppm 10.62, 11.46, 16.06, 16.55, 18.98, 19.03, 19.17, 21.28, 21.52, 24.32, 24.63, 25.99, 26.57, 26.59, 27.25, 28.90, 28.94, 32.52, 32.98, 33.23, 40.23, 41.46, 46.16, 46.90, 171.59, 171.68, 171.94, 174.51, 174.70.

### Example 11: Synthesis of 3,5-dimethylcyclohexan-1-one oxime

To a one neck round bottom flask, was added the 10.2 g (81 mmol) of 3,5-dimethylcyclohexan-1-one, ethanol (125 mL), and 12.8 g (162 mmol) pyridine. While stirring at room temperature under nitrogen, 6.2 g (89 mmol) of hydroxylamine HCl was added portion wise. The reaction continued to stir until completion as monitored by gas chromatography. The solvent was removed under reduced pressure. The residue was then diluted with ethyl acetate and washed with H₂O (1×), 1M HCl (1×) and brine (1×). The aqueous layer was back extracted with ethyl acetate (1×). The organics were combined and dried over MgSO₄, filtered, and concentrated under reduced pressure. The crude material was purified via automated silica gel chromatography (Biotage Isolera, 6-50% ethyl acetate/hexane) to afford 11.12 g (97%) of 3,5-dimethylcyclohexan-1-one oxime as a solid. Odor characteristics: green, camphoraceous, earthy, floral, grassy, caryophyllene-like.

1H NMR δ ppm 0.81 (m, 1H) 0.97 (m, 6H) 1.23 (m, 1H) 1.59 (m, 3H) 1.73 (m, 1H) 2.32 (dt, J=9.62, 2.06 Hz, 1H) 3.31 (dt, J=13.97, 1.95 Hz, 1H). 13C NMR δ ppm 22.30, 22.52, 31.98, 32.10, 33.24, 39.92, 43.53, 160.87.

### Example 12: Synthesis of 3,5-dimethylcyclohexan-1-one O-allyl oxime

To an oven dried, two neck round bottom flask was added 0.476 g (19.8 mmol) sodium hydride and anhydrous THE (20 mL). The grey suspension was stirred under nitrogen, then cooled to 0° C. in an ice bath. A solution of 1.40 g (9.9 mmol) 3,5-dimethylcyclohexan-1-one oxime in anhydrous THE (10 mL) was added dropwise to the suspension at this temperature. After addition, the ice bath was removed to let the mixture warm to room temperature, where it remained for 2 hours. At this temperature, 0.9 mL (10.9 mmol) allyl bromide was added dropwise. After addition, the reaction continued to stir at room temperature until the reaction was complete by GC. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 2-40% Ethyl Acetate/Hexanes) with final isolation by Kugelrohr distillation to afford 0.915 g (50.8%) of 3,5-dimethylcyclohexan-1-one O-allyl oxime as an oil. Odor characteristics: solventy.

1H NMR δ ppm 0.81 (m, 1H) 0.95 (m, 6H) 1.23 (m, 1H) 1.59 (m, 3H) 1.72 (m, 1H) 2.32 (dt, J=9.50, 1.89 Hz, 1H) 3.25 (m, 1H) 4.50 (dt, J=5.50, 1.37 Hz, 2H) 5.17 (m, 1H) 5.27 (m, 1H) 5.97 (m, 1H). 13C NMR δ ppm 22.31, 22.53, 32.20, 32.93, 33.38, 39.97, 43.52, 74.16, 117.13, 134.71, 160.47.

### Example 13: Synthesis of 3,5-dimethylcyclohexan-1-one O-ethyl oxime

To an oven dried, two neck round bottom flask was added 0.476 g (19.8 mmol) sodium hydride and anhydrous THE (20 mL). The grey suspension was stirred under nitrogen, then cooled to 0° C. in an ice bath. A solution of 1.23 g (8.7 mmol) 3,5-dimethylcyclohexan-1-one oxime in anhydrous THE (10 mL) was added dropwise to the suspension at this temperature. After addition, the ice bath was removed to let the mixture warm to room temperature, where it remained for 2 hours. At this temperature, 0.9 mL (10.9 mmol) iodoethane was added dropwise. After addition, the reaction continued to stir at room temperature until the reaction was complete by GC. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 2-40% Ethyl Acetate/Hexanes) to afford 0.180 g (12.2%) of 3,5-dimethylcyclohexan-1-one O-ethyl oxime as an oil. Odor characteristics: solventy, strawberry, sweet, harsh and chemical.

1H NMR δ ppm 0.79 (d, J=12.82 Hz, 1H) 0.97 (m, 6H) 1.22 (m, 4H) 1.59 (m, 3H) 1.71 (m, 1H) 2.31 (dt, J=9.50, 1.89 Hz, 1H) 3.22 (m, 1H) 4.03 (q, J=7.02 Hz, 2H). 13C NMR δ ppm 14.65, 22.34, 22.54, 32.19, 32.85, 33.38, 40.06, 43.56, 68.70, 159.86.

### Example 14: Synthesis of 3,5-dimethylcyclohexan-1-one O-(3-hydroxypropyl) oxime

To an oven dried, two neck round bottom flask, equipped with a thermometer and an addition funnel, was added 0.667 g (27 mmol) sodium hydride and anhydrous THE (50 mL). The grey suspension was under nitrogen, then cooled to 0° C. in an ice bath. A solution of 1.87 g (13 mmol) 3,5-dimethylcyclohexan-1-one oxime in anhydrous THE (10 mL) was added dropwise to the suspension at this temperature. After addition, the ice bath was removed to let the mixture warm to room temperature, where it remained for 2 hours. At this temperature, 1.41 mL (16 mmol) of 3-bromopropan-1-ol was added dropwise. After addition, the reaction continued to stir at room temperature until the reaction was complete by GC. The mixture was cooled to 0° C. in an ice bath, diluted with diethyl ether and quenched with water. The layers were separated, and the aqueous layer was back extracted with diethyl ether (3×). The organics were combined, dried over MgSO₄, filtered, and concentrated under reduced pressure. The crude material was purified via automated silica gel chromatography (Biotage Isolera, 5-40% Ethyl Acetate/Hexanes) with final isolation by Kugelrohr distillation (83-86° C. @12 Torr) to afford 0.35 g (13.3%) of 3,5-dimethylcyclohexan-1-one O-(3-hydroxypropyl) oxime as an oil. Odor characteristics: floral, hint of green, butyric.

1H NMR δ ppm 0.79 (q, 1H) 0.95 (t, 6H) 1.23 (t, 1H) 1.57 (m, 4H) 1.73 (d, 1H) 1.89 (quin, 2H) 2.30 (dt, 1H) 2.40 (br. s, 1H) 3.17 (m, 1H) 3.73 (br. m, 2H) 4.15 (t, 2H). 13C NMR δ ppm 22.27, 22.51, 32.23, 32.49, 32.89, 33.37, 39.97, 43.47, 60.81, 71.13, 160.54.

### Example 15: Synthesis of 3,5-dimethylcyclohexan-1-one O-acetyl oxime

To an oven dried, round bottom flask was added 1.87 g (13 mmol) of 3,5-dimethylcyclohexan-1-one oxime and anhydrous DCM (50 mL). 2.22 mL (16 mmol) of triethylamine was added to the flask and stirred under nitrogen. The flask was cooled to 0° C. where 1.04 mL (15 mmol) of acetyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (90° C. @1.4 Torr) to afford 1.273 g (52.6%) of 3,5-dimethylcyclohexan-1-one O-acetyl oxime as an oil. Odor characteristics: hot cinnamon, rose, minty, clean.

1H NMR δ ppm 0.84 (q, 1H) 0.98 (m, 6H) 1.38 (m, 1H) 1.67 (m, 4H) 2.13 (s, 3H) 2.56 (m, 1H) 3.18 (m, 1H). 13C NMR δ ppm 19.80, 22.21, 22.40, 32.46, 33.35, 34.34, 39.71, 43.13, 168.26, 169.10.

### Example 16: Synthesis of 3,5-dimethylcyclohexan-1-one O-propionyl oxime

To an oven dried, round bottom flask was added 1.86 g (13 mmol) of 3,5-dimethylcyclohexan-1-one oxime and anhydrous DCM (50 mL). Triethylamine (2.19 mL, 16 mmol) was added to the flask and stirred under nitrogen. The flask was cooled to 0° C. where 1.27 mL (15 mmol) of propionyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (100° C. @0.6 Torr) to afford 1.872 g (72.0%) of 3,5-dimethylcyclohexan-1-one O-propionyl oxime as an oil. Odor characteristics: fresh, earthy, green, rosy, geranium, natural, mid-strength.

1H NMR δ ppm 0.84 (m, 1H) 0.98 (m, 6H) 1.18 (t, 3H) 1.37 (m, 1H) 1.71 (m, 4H) 2.41 (q, 2H) 2.57 (m, 1H) 3.17 (m, 1H). 13C NMR δ ppm 9.22, 22.21, 22.42, 26.50, 32.42, 33.32, 34.31, 39.75, 43.14, 168.25, 172.36.

### Example 17: Synthesis of 3,5-dimethylcyclohexan-1-one O-isobutyryl oxime

To an oven dried, round bottom flask was added 1.86 g (13 mmol) of 3,5-dimethylcyclohexan-1-one oxime and anhydrous DCM (50 mL). Triethylamine (2.19 mL, 16 mmol) was added to the flask and stirred under nitrogen. The flask was cooled to 0° C. where 1.52 mL (15 mmol) of isobutyryl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (|×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (100° C. @0.56 Torr) to afford 2.149 g (77.2%) of 3,5-dimethylcyclohexan-1-one O-isobutyryl oxime as an oil. Odor characteristics: weak.

1H NMR δ ppm 0.84 (m, 1H) 0.99 (dd, 6H) 1.20 (d, 6H) 1.40 (m, 1H) 1.69 (m, 4H) 2.62 (m, 2H) 3.17 (m, 1H). 13C NMR δ ppm 19.1, 22.23, 22.47, 32.40, 33.22, 33.28, 34.31, 39.79, 43.16, 168.54, 174.68.

### Example 18: Synthesis of 3,5-dimethylcyclohexan-1-one O-cyclopropanecarbonyl oxime

To an oven dried, round bottom flask was added 1.87 g (13 mmol) of 3,5-dimethylcyclohexan-1-one oxime and anhydrous DCM (50 mL). Triethylamine (2.22 mL, 16 mmol) was added to the flask and stirred under nitrogen. The flask was cooled to 0° C. where 1.34 mL (15 mmol) of cyclopropanecarbonyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 6-50% ethyl acetate/hexane) with final isolation by Kugelrohr distillation (110-113° C. @0.16 Torr) to afford 2.185 g (78.8%) of 3,5-dimethylcyclohexan-1-one O-cyclopropanecarbonyl oxime as an oil. Odor characteristics: weak.

1H NMR δ ppm 0.92 (m, 3H) 1.01 (dd, 6H) 1.08 (m, 2H) 1.40 (m, 1H) 1.68 (m, 5H) 2.57 (m, 1H) 3.23 (m, 1H). 13C NMR δ ppm 8.84, 11.65, 22.23, 22.45, 32.44, 33.31, 34.55, 39.74, 43.17, 168.03, 172.91.

### Example 19: Synthesis of 3,5-dimethylcyclohexan-1-one O-cyclohexanecarbonyl oxime

To an oven dried, round bottom flask was added 1.87 g (13 mmol) of 3,5-dimethylcyclohexan-1-one oxime and anhydrous DCM (50 mL). Triethylamine (2.22 mL, 16 mmol) was added to the flask and stirred under nitrogen. The flask was cooled to 0° C. where 1.98 mL (15 mmol) of cyclohexanecarbonyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 6-50% ethyl acetate/hexane) with final isolation by Kugelrohr distillation (110-113° C. @0.16 Torr) to afford 1.611 g (48.3%) of 3,5-dimethylcyclohexan-1-one O-cyclohexanecarbonyl oxime as an oil. Odor characteristics: strong, fruity, fermented apple.

1H NMR δ ppm 0.83 (m, 1H) 1.01 (dd, 6H), 1.30 (m, 3H), 1.39 (m, 1H) 1.68 (m, 10H) 1.94 (m, 2H) 2.40 (m, 1H) 2.59 (m, 1H) 3.16 (m, 1H). 13C NMR δ ppm 22.23, 22.48, 25.54, 25.76, 29.08, 32.40, 33.27, 34.34, 39.81, 42.47, 43.17, 168.47, 173.62.

### Example 20: Synthesis of 4-(tert-pentyl)cyclohexan-1-one oxime

To a one neck round bottom flask, was added the 13.77 g (82 mmol) of 4-(tert-pentyl)cyclohexan-1-one, ethanol (125 mL) and 12.97 g (164 mmol) pyridine. While stirring at room temperature under nitrogen, 6.25 g (90 mmol) of hydroxylamine HCl was added portion wise. The reaction was stirred under nitrogen, at room temperature until the reaction was completed by gas chromatography. The solvent was removed under reduced pressure. The residue was then diluted with ethyl acetate and washed with H₂O (1×), 1N HCl (1×) and brine (1×). The aqueous layer was back extracted with ethyl acetate (1×). The organics were combined and dried over MgSO₄, filtered and concentrated under reduced pressure. The crude material was purified via automated silica gel chromatography (Biotage Isolera, 5-40% ethyl acetate/hexane) to afford 13.43 g (89.5%) of 4-(tert-pentyl)cyclohexan-1-one oxime as a solid. Odor characteristics: weak.

1H NMR δ ppm 0.80 (m, 9H) 1.25 (m, 5H) 1.59-1.70 (m, 2H) 1.88 (m, 2H) 2.05 (m, 1H) 2.41 (m, 1H) 3.36 (m, 1H) 7.25 (s, 1H). 13C NMR δ ppm 8.20, 24.29, 24.35, 26.02, 27.27, 32.15, 32.79, 34.86, 44.83, 161.41.

### Example 21: Synthesis of 4-(tert-pentyl)cyclohexan-1-one O-methyl oxime

To a one neck round bottom flask, was added the 2.0 g (11.9 mmol) of 4-(tert-pentyl)cyclohexan-1-one, ethanol (25 mL) and 1.9 mL (23.8 mmol) pyridine. While stirring at room temperature under nitrogen, 1.09 g (13.1 mmol) of O-methylhydroxylamine HCl was added portion wise. The reaction was stirred under nitrogen, at room temperature until the reaction was completed by gas chromatography. The solvent was removed under reduced pressure. The residue was then diluted with ethyl acetate and washed with H₂O (1×), 1N HCl (1×) and brine (1×). The aqueous layer was back extracted with ethyl acetate (1×). The organics were combined and dried over MgSO₄, filtered and concentrated under reduced pressure. The crude material was purified via automated silica gel chromatography (Biotage Isolera, 5-40% ethyl acetate/hexane) to afford 1.864 g (79.7%) of 4-(tert-pentyl)cyclohexan-1-one O-methyl oxime as a solid. Odor characteristics: weak.

1H NMR δ ppm 0.78 (m, 9H) 1.25 (q, J=7.33 Hz, 5H) 1.67 (s, 1H) 1.84 (m, 2H) 2.03 (m, 1H) 2.39 (m, 1H) 3.23 (ddt, J=14.37, 4.29, 2.35, 2.35 Hz, 1H) 3.79 (s, 3H). 13C NMR δ ppm 8.20, 24.35, 25.10, 26.12, 27.33, 32.18, 32.78, 34.85, 44.79, 61.07, 160.51.

### Example 22: Synthesis of 4-(tert-pentyl)cyclohexan-1-one O-allyl oxime

To an oven dried, two neck round bottom flask was added 0.48 g (20 mmol) sodium hydride and anhydrous THE (50 mL). The grey suspension was stirred under nitrogen, then cooled to 0° C. in an ice bath. A solution of 1.8 g (10 mmol) 4-(tert-pentyl)cyclohexan-1-one oxime in anhydrous THE (10 mL) was added dropwise to the suspension at this temperature. After addition, the ice bath was removed to let the mixture warm to room temperature, where it remained for 2 hours. At this temperature, 1.33 mL (11 mmol) allyl bromide was added dropwise. After addition, the reaction continued to stir at room temperature until the reaction was complete by GC. The mixture was cooled to 0° C. in an ice bath, diluted with diethyl ether and quenched with water. The layers were separated, and the aqueous layer was back extracted with diethyl ether (3×). The organics were combined, washed with saturated Na₂S₂O₃, dried over MgSO₄, filtered, and concentrated under reduced pressure. The crude material was purified via automated silica gel chromatography (Biotage Isolera, 5-40% Ethyl Acetate/Hexanes) with final isolation by Kugelrohr distillation (84-86° C. @0.2 Torr) to afford 0.821 g (37.4%) of 4-(tert-pentyl)cyclohexan-1-one O-allyl oxime as an oil. Odor characteristics: pineapple, burnt, white wine, sour, damp, syrup.

1H NMR δ ppm 0.79 (m, 9H) 1.27 (m, 5H), 1.69 (td, 1H) 1.85 (m, 2H) 2.03 (td, 1H) 2.44 (m, 1H) 3.31 (m, 1H) 4.50 (d, 2H) 5.18 (dd, 1H) 5.27 (dd, 1H) 5.99 (m, 1H). 13C NMR δ ppm 8.20, 24.34, 25.32, 26.11, 27.35, 32.17, 32.78, 34.85, 44.84, 74.15, 117.06, 134.73, 160.78.

### Example 23: Synthesis of 4-(tert-pentyl)cyclohexan-1-one O-acetyl oxime

To an oven dried, round bottom flask was added 1.81 g (10 mmol) of 4-(tert-pentyl)cyclohexan-1-one oxime and anhydrous DCM (50 mL). Triethylamine (1.67 mL, 12 mmol) was added to the flask and stirred under nitrogen. The flask was cooled to 0° C. where 0.78 mL (11 mmol) of acetyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (82-85° C. @0.2 Torr) to afford 1.458 g (65.5%) of 4-(tert-pentyl)cyclohexan-1-one O-acetyl oxime as an oil. Odor characteristics: amber, orris, floral, woody.

1H NMR δ ppm 0.78 (m, 9H), 1.24 (m, 5H), 1.85 (m, 3H) 2.12 (m, 4H) 2.64 (m, 1H) 3.48 (m, 1H). 13C NMR δ ppm 8.17, 19.76, 24.28, 26.25, 26.84, 27.16, 32.07, 32.73, 34.84, 44.55, 168.78, 169.04.

### Example 24: Synthesis of 4-(tert-pentyl)cyclohexan-1-one O-propionyl oxime

To an oven dried, round bottom flask was added 1.81 g (10 mmol) of 4-(tert-pentyl)cyclohexan-1-one oxime and anhydrous DCM (50 mL). Triethylamine (1.67 mL, 12 mmol) was added to the flask and stirred under nitrogen. The flask was cooled to 0° C. where 0.96 mL (11 mmol) of acetyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (82-85° C. @0.2 Torr) to afford 1.768 g (74.6%) of 4-(tert-pentyl)cyclohexan-1-one O-propionyl oxime as a solid. Odor characteristics: weak.

1H NMR δ ppm 0.80 (m, 9H) 1.23 (m, 8H) 1.86 (m, 3H) 2.13 (m, 1H) 2.41 (q, 2H) 2.67 (m, 1H) 3.25 (m, 1H). 13C NMR δ ppm 8.18, 9.24, 24.30, 26.24, 26.54, 26.82. 27.15, 32.12, 32.75, 34.85, 44.58, 168.82, 172.32.

### Example 25: Synthesis of 4-(tert-pentyl)cyclohexan-1-one O-isobutyryl oxime

To an oven dried, round bottom flask was added 1.81 g (10 mmol) of 4-(tert-pentyl)cyclohexan-1-one oxime and anhydrous DCM (50 mL). Triethylamine (1.67 mL, 12 mmol) was added to the flask and stirred under nitrogen. The flask was cooled to 0° C. where 1.15 mL (11 mmol) of isobutyryl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (107° C. @0.18 Torr) to afford 1.878 g 74.8%) of 4-(tert-pentyl)cyclohexan-1-one O-isobutyryl oxime as an oil. Odor characteristics: cheesy.

1H NMR δ ppm 0.77 (m, 9H) 1.21 (m, 11H) 1.85 (m, 3H) 2.10 (m, 1H) 2.61 (m, 2H) 3.21 (m, 1H). 13C NMR δ ppm 8.16, 19.10, 24.28, 26.19, 26.76, 27.07, 32.10, 32.72, 33.24, 34.83, 44.54, 169.03, 174.58.

### Example 26: Synthesis of 4-(tert-pentyl)cyclohexan-1-one O-cyclopropanecarbonyl oxime

To an oven dried, round bottom flask was added 1.81 g (10 mmol) of 4-(tert-pentyl)cyclohexan-1-one oxime and anhydrous DCM (50 mL). Triethylamine (1.67 mL,12 mmol) was added to the flask and stirred under nitrogen. The flask was cooled to 0° C. where 1.01 mL (11 mmol) of cyclopropanecarbonyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (121-123° C. @0.15 Torr) to afford 1.494 g (60.0%) of 4-(tert-pentyl)cyclohexan-1-one O-cyclopropanecarbonyl oxime as an oil. Odor characteristics: weak.

1H NMR δ ppm 0.79 (m, 9H) 0.90 (m, 2H) 1.08 (m, 2H) 1.26 (m, 5H) 1.66 (m, 1H) 1.87 (m, 3H) 2.12 (m, 1H) 2.71 (m, 1H) 3.29 (m, 1H). 13C NMR δ ppm 8.19, 8.80, 11.67, 24.31, 26.25, 26.84, 27.14, 32.09, 32.75, 34.85, 44.57, 168.58, 172.88.

### Example 27: Synthesis of 4-(tert-pentyl)cyclohexan-1-one O-cyclohexanecarbonyl oxime

To an oven dried, round bottom flask was added 1.81 g (10 mmol) of 4-(tert-pentyl)cyclohexan-1-one oxime and anhydrous DCM (50 mL). Triethylamine (1.67 mL,12 mmol) was added to the flask and stirred under nitrogen. The flask was cooled to 0° C. where 1.50 mL (11 mmol) of cyclohexanecarbonyl chloride was added dropwise to the mixture. After addition, the ice bath was removed, and the mixture warmed to room temperature where it remained until completion. The cloudy mixture was diluted with DCM until the precipitate dissolved. The mixture was then washed with H₂O (1×), sat. NaHCO₃(1×), and H₂O (1×). The organic layer was separated, dried over MgSO₄, filtered, and concentrated under reduced pressure. The crude product was then isolated by automated silica gel chromatography (Biotage Isolera, 5-40% ethyl acetate/hexane) with final isolation by Kugelrohr distillation (135° C. @0.15 Torr) to afford 1.527 g (52.5%) of 4-(tert-pentyl)cyclohexan-1-one O-cyclohexanecarbonyl oxime as an oil. Odor characteristics: weak.

1H NMR δ ppm 0.80 (m, 9H) 1.26 (m, 8H) 1.52 (q, 2H) 1.60 (m, 2H) 1.73-1.99 (m, 7H) 2.13 (m, 1H) 2.67 (m, 1H) 3.24 (m, 1H). 13C NMR δ ppm 8.18, 24.28, 24.31, 25.53, 25.77, 26.21, 26.84, 27.09, 29.13, 32.15, 32.75, 34.85, 42.54, 44.58, 169.03, 173.58.

### Example 28: Fine fragrance composition

A fresh citrus type fragrance composition was prepared using the compound of Example 1 and other fragrance compounds of various fragrance categories. The composition is provided in Table 1.

TABLE 1

| Fragrance category | % |
|---|---|
| Aldehydic | 0.044 |
| Citrus | 14.30 |
| Floral | 49.511 |
| Fruity | 0.066 |
| Green | 1.815 |
| Gourmand | 0.165 |
| Herbal | 0.957 |
| Marine | 0.154 |
| Mossy | 0.055 |
| Musk | 4.829 |
| Piney | 0.022 |
| Spicy | 0.715 |
| Woody/amber | 13.695 |
| Solvent | 12.672 |
| Compound of Example 1 @ 10% in DPG | 1.000 |
| Total | 100 |

The fragrance composition with and without the compound of Example 1 was evaluated by 1 expert Perfumer, who found that adding the compound of Example 1 to the composition amplified the existing citrus jasmine floral notes of the composition.

### Example 29: Fine fragrance composition

A tea orange blossom type fragrance composition was prepared using the compound of Example 1 and other fragrance compounds of various fragrance categories. The composition is provided in Table 2.

TABLE 2

| Fragrance category | % |
|---|---|
| Animalic | 1.98 |
| Citrus | 17.34 |
| Floral | 38.06 |
| Fruity | 2.08 |
| Green | 0.50 |
| Herbaceous | 0.30 |
| Mossy | 0.69 |
| Musk | 15.86 |
| Spicy | 0.20 |
| Woody/amber | 22.89 |
| Compound of Example 1 @ 1% TEC | 0.10 |
| Total | 100 |

The fragrance composition with and without the compound of Example 1 was evaluated by 1 expert Perfumer, who found that adding the compound of Example 1 to the composition enhanced the floralcy and citrus effect and gave the petitgrain (comprised within citrus category) a quite natural effect.

### Example 30: Fragrance composition for use in a shampoo

A floral powdery type fragrance composition was prepared using the compound of Example 1 and other fragrance compounds of various fragrance categories to demonstrate its use in a shampoo. The composition is provided in Table 3.

TABLE 3

| Fragrance category | % |
|---|---|
| Citrus | 3.10 |
| Floral | 68.31 |
| Fruity | 1.53 |
| Green | 0.01 |
| Herbal | 2.10 |
| Musk | 20.00 |
| Powdery | 1.60 |
| Spicy | 0.20 |
| Woody/amber | 0.80 |
| Solvent | 1.85 |
| Compound of Example 1 @ 10% TEC | 0.50 |
| Total | 100 |

A second fragrance composition as shown by Table 3 was prepared with 5-methyl-3-heptanone oxime (available commercially as STEMONE®) instead of using the compound of Example 1. Each of the fragrance compositions was dosed at 0.5% into a commercial shampoo base and evaluated by 1 Perfumers and 2 experts in the field of fragrance evaluations, who found that adding the compound of Example 1 to the composition provided pleasant natural green crispy note compared to the second fragrance composition.

### Example 31: Fragrance composition for use in a shower gel

A floral fruity type fragrance composition was prepared using the compound of Example 1 and other fragrance compounds of various fragrance categories to demonstrate its use in a shower gel. The composition is provided in Table 4.

TABLE 4

| Fragrance category | % |
|---|---|
| Citrus | 1.5 |
| Floral | 48.8 |
| Fruity | 3.8 |
| Gourmand | 1.0 |
| Green | 1.3 |
| Herbal | 1.6 |
| Musk | 0.2 |
| Powdery | 1.5 |
| Spicy | 0.2 |
| Woody/amber | 22.9 |
| Solvent | 16.9 |
| Compound of Example 1 | 0.5 |
| Total | 100 |

The fragrance composition was dosed at 1% into a commercial shower gel and evaluated by 1 Perfumer and 2 experts in the field of fragrance evaluations. The fragrance had a nice green cassis character.

### Example 32: Fragrance Composition for Use in Fabric Softener

A fragrance composition was prepared using the compound of Example 1 and other fragrance compounds of various fragrance categories to demonstrate its use in a fabric softener. The composition is provided in Table 5.

TABLE 5

| Fragrance category | % |
|---|---|
| Citrus | 10 |
| Floral | 25.9 |
| Fruity | 15.6 |
| Green | 5.6 |
| Herbal | 15.4 |
| Musk | 4 |
| Spicy | 1.4 |
| Woody/amber | 20.1 |
| Solvent | 1.8 |
| Compound of Example 1 | 0.2 |
| Total | 100 |

A second fragrance composition as shown by Table 5 was prepared without the compound of Example 1. Each of the fragrance compositions was dosed at 1% into a commercial fabric softener and evaluated by 1 Perfumer and 2 experts in the field of fragrance evaluations, who found that adding the compound of Example 1 to the composition created a very natural leafy green vegetal top note, and provided clean crisp strength throughout all stages in fabric evaluation, compared to the fragrance composition without the compound of Example 1.

### Example 33: Fine fragrance composition

A floral, woody type fragrance composition was prepared using the compound of Example 2 and other fragrance compounds of various fragrance categories. The composition is provided in Table 6.

TABLE 6

| Fragrance category | % |
|---|---|
| Animalic | 2.1 |
| Balsamic | 12.05 |
| Citrus | 0.5 |
| Floral | 57.31 |
| Fruity | 0.02 |
| Green | 0.02 |
| Musk | 21 |
| Woody/amber | 6 |
| Compound of Example 2 | 1 |
| Total | 100 |

### Example 34: Fine Fragrance Composition

A floral feminine fragrance composition was prepared using the compound of Example 2 and other fragrance compounds of various fragrance categories. The composition is provided in Table 7.

TABLE 7

| Fragrance category | % |
|---|---|
| Animalic | 2.11 |
| Balsamic | 12.11 |
| Citrus | 0.50 |
| Floral | 57.60 |
| Fruity | 0.02 |
| Green | 0.02 |
| Musk | 21.11 |
| Woody/amber | 6.03 |
| Compound of Example 2 | 0.50 |
| Total | 100 |

### Example 35: A Fine Fragrance Composition

A fruity rose type fragrance composition was prepared using the compound of Example 2 and other fragrance compounds of various fragrance categories. The composition is provided in Table 8.

TABLE 8

| Fragrance category | % |
|---|---|
| Animalic | 0.2 |
| Citrus | 2.5 |
| Floral | 32.5 |
| Fruity | 2.8 |
| Green | 0.3 |
| Herbal | 0.5 |
| Musk | 21.4 |
| Woody/amber | 39.7 |
| Compound of Example 2 @ 1% DPG | 0.1 |
| Total | 100 |

### Example 36: Fragrance Composition for a Use in Shower Gel

A floral fruity type fragrance composition was prepared using the compound of Example 2 and other fragrance compounds of various fragrance categories. The composition is provided in Table 9.

TABLE 9

| Fragrance category | % |
|---|---|
| Citrus | 1.5 |
| Floral | 48.75 |
| Fruity | 3.8 |
| Gourmand | 1.2 |
| Green | 1.45 |
| Herbal | 1.55 |
| Musk | 0.2 |
| Spicy | 1.5 |
| Powdery | 0.15 |
| Woody/amber | 22.85 |
| Solvent | 16.55 |
| Compound of Example 2 | 0.5 |
| Total | 100 |

### Example 37: Fragrance Composition for Dish Detergent

A grapefruit citrus type fragrance composition was prepared using the compound of Example 2 and other fragrance compounds of various fragrance categories. The composition is provided in Table 10.

TABLE 10

| Fragrance category | % |
|---|---|
| Aldehydic | 0.2 |
| Citrus | 70.4 |
| Floral | 13.6 |
| Fruity | 2.4 |
| Green | 0.2 |
| Herbal | 0.4 |
| Piney | 2.4 |
| Woody/Amber | 0.4 |
| Solvent | 8 |
| Compound of Example 2 | 2 |
| Total | 100 |

A second fragrance composition as shown by Table 10 was prepared with ethyl tricyclo[5.2.1.0^{20.6}]decan-2 carboxylate (available commercially as Fruitate®) instead of using the compound of Example 2. Each of the fragrance compositions was dosed at 2% into an unscented commercial dish detergent and evaluated by 7 Perfumers and 5 experts in the field of fragrance evaluations, who found that adding the compound of Example 2 to the composition provided more volume, and natural fruity freshness compared to the second fragrance composition.

### Example 38: Fragrance Composition for Laundry Detergent

A fruity floral type fragrance composition was prepared using the compound of Example 2 and other fragrance compounds of various fragrance categories. The composition is provided in Table 11.

TABLE 11

| Fragrance category | % |
|---|---|
| Aldehydic | 0.8 |
| Citrus | 6 |
| Floral | 36.6 |
| Fruity | 24.8 |
| Green | 2.6 |
| Gourmand | 0.2 |
| Herbal | 8 |
| Musk | 10 |
| Spicy | 1 |
| Woody/Amber | 6 |
| Solvent | 2 |
| Compound of Example 2 | 2 |
| Total | 100 |

A second fragrance composition as shown by Table 11 was prepared with ethyl tricyclo[5.2.1.0^{20.6}]decan-2 carboxylate instead of using the compound of Example 2. Each of the fragrance compositions was dosed at 2% into an unscented commercial laundry detergent and evaluated by 7 Perfumers and 5 experts in the field of fragrance evaluations, who found that adding the compound of Example 2 to the composition provided much more fresh fruity lift and volume to the fragrances, as well as introducing a more natural, zesty character and a hedonic multifaceted fruitiness compared to the second fragrance composition.

### Example 39: Fragrance Composition for Fabric Softener

A fruity floral type fragrance composition was prepared using the compound of Example 2 and other fragrance compounds of various fragrance categories. The composition is provided in Table 11 shown in Example 38.

A second fragrance composition as shown by Table 11 was prepared with ethyl tricyclo[5.2.1.0^{20.6}]decan-2 carboxylate instead of using the compound of Example 2. Each of the fragrance compositions was dosed at 2% into a unscented commercial fabric softener and evaluated by 7 Perfumers and 5 experts in the field of fragrance evaluations, who found that adding the compound of Example 2 to the composition provided much more fresh multifaced fruity notes on top compared to the second fragrance composition.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1-43. (canceled)

44. A compound of Formula (II), or isomer thereof: 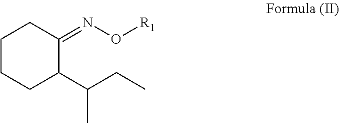
wherein
R₁ is a C1-C5 linear or branched alkyl group, C2-C5 linear or branched alkenyl group, C1-C5 linear or branched alcohol group, or C(O)R₂, and
R₂ is a C1-C5 linear or branched alkyl group.

45. The compound of claim 44, wherein the compound of Formula (II) is selected from the group consisting of:
2-(sec-butyl)cyclohexan-1-one O-methyl oxime,
2-(sec-butyl)cyclohexan-1-one O-allyl oxime,
2-(sec-butyl)cyclohexan-1-one O-ethyl oxime,
2-(sec-butyl)cyclohexan-1-one 0-(3-methylbut-2-en-1-yl) oxime,
2-(sec-butyl)cyclohexan-1-one O-butyl oxime,
2-(sec-butyl)cyclohexan-1-one 0-(3-hydroxypropyl) oxime,
2-(sec-butyl)cyclohexan-1-one 0-acetyl oxime,
2-(sec-butyl)cyclohexan-1-one O-propionyl oxime, and
2-(sec-butyl)cyclohexan-1-one O-isobutyryl oxime.

46. A flavor or fragrance composition comprising a compound of Formula (II), or isomer thereof: 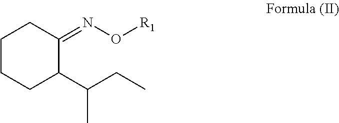
wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.

47. The flavor or fragrance composition of claim 46, wherein the compound of Formula (II) is selected from the group consisting of:
2-(sec-butyl)cyclohexan-1-one oxime,
2-(sec-butyl)cyclohexan-1-one O-methyl oxime,
2-(sec-butyl)cyclohexan-1-one O-allyl oxime,
2-(sec-butyl)cyclohexan-1-one O-ethyl oxime,
2-(sec-butyl)cyclohexan-1-one 0-(3-methylbut-2-en-1-yl) oxime,
2-(sec-butyl)cyclohexan-1-one O-butyl oxime,
2-(sec-butyl)cyclohexan-1-one 0-(3-hydroxypropyl) oxime,
2-(sec-butyl)cyclohexan-1-one O-acetyl oxime,
2-(sec-butyl)cyclohexan-1-one O-propionyl oxime, and
2-(sec-butyl)cyclohexan-1-one O-isobutyryl oxime.

48. The flavor or fragrance composition of claim 46, wherein composition comprises at least one compound of Formula (II).

49. The flavor or fragrance composition of claim 46, wherein the composition is a fragrance composition.

50. The flavor or fragrance composition of claim 49, wherein the fragrance composition comprises at least one compound in a concentration of from about 0.001% to about 20% by weight of the fragrance composition.

51. The flavor or fragrance composition of claim 49, wherein the fragrance composition comprises at least one compound in a concentration of from about 0.01% to about 10% by weight of the fragrance composition.

52. The flavor or fragrance composition of claim 49, wherein the fragrance composition further comprises at least one compounds selected from the group consisting of one or more aldehydic compound(s), animalic compound(s), balsamic compound(s), citrus compound(s), floral compound(s), fruity compound(s), gourmand compound, green compound(s), herbaceous compound(s), marine compound(s), mossy compound(s), musk compound(s), piney compound(s), powdery compound(s), spicy compound(s), and/or woody/amber compound(s).

53. The flavor or fragrance composition of claim 49, wherein the fragrance composition further comprises a fragrance carrier or a fragrance base.

54. The flavor or fragrance composition of claim 49, wherein the fragrance composition can be in the form of a simple mixture or a biphasic system.

55. The flavor or fragrance composition of claim 54, wherein the biphasic system is an emulsion or microemulsion.

56. The flavor or fragrance composition of claim 49, wherein the fragrance composition can be used in consumer products selected form the group consisting of air care products, fine fragrances, personal care products, cosmetic, fabric and home care products, pharmaceutical and over-the-counter products, insect repellents or insecticides, and pet care products.

57. The flavor or fragrance composition of claim 46, wherein the composition is a flavor composition.

58. The flavor or fragrance composition of claim 57, wherein the flavor composition comprises at least one compound in a concentration of from about 0.0005% to about 20% by weight of the flavor composition.

59. The flavor or fragrance composition of claim 57, wherein the flavor composition further comprises at least one additional flavor composition or flavor compound.

60. The flavor or fragrance composition of claim 57, wherein the flavor composition further comprises a flavor carrier.

61. The flavor or fragrance composition of claim 57, wherein the flavor composition further comprises at least one material selected from the group consisting of one or more of solvents, support materials, diluents, carriers, preservatives, flavor compounds, flavor enhancers, antioxidants, color retention agents, dyes or lakes, sequestrants, emulsifiers, stabilizers, acids, bases, and anticaking agents.

62. The flavor or fragrance composition of claim 57, wherein the flavor composition can be in a simple mixture or an encapsulated form.

63. A method of modifying the odor properties of a fragrance composition by adding to the fragrance composition an olfactory effective quantity of a fragrance compound of Formula (II), or isomer thereof: 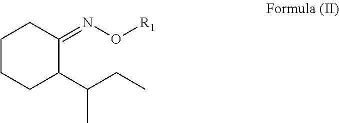
wherein
R₁ is a hydrogen atom, C1-C5 linear or branched alkyl group, C2-C5 linear or branched alkenyl group, C1-C5 linear or branched alcohol group, or C(O)R₂, and
R₂ is a C1-C5 linear or branched alkyl group.
